(19) United States
(12) Patent Application Publication
(10) Pub. No.: US 2026/0021109 A1
(43) Pub. Date: Jan. 22, 2026

(54) PROSTANOID RECEPTOR AGONISTS AS NON-TUBERCULOUS ANTI-MYCOBACTERIAL AGENTS

(71) Applicant: Chariot Innovations Limited, London (GB)

(72) Inventors: Robin BASU ROY, London (GB); Serge MOSTOWY, London (GB)

(21) Appl. No.: 19/116,732

(22) PCT Filed: Sep. 28, 2023
(86) PCT No.: PCT/GB2023/052516

§ 371 Date: Mar. 28, 2025

(30) Foreign Application Priority Data
Sep. 28, 2022 (GB) 2214206.1

(51) Int. Cl. A61K 31/5585; A61K 31/133; A61K 31/27; A61K 31/343; A61K 31/407; A61K 31/4406; A61K 31/4409; A61K 31/496; A61K 31/4965; A61K 31/5575; A61K 31/5578; A61K 31/7036; A61K 31/7048; A61K 31/7052; A61P 31/04

(52) U.S. Cl. CPC A61K 31/5585; A61K 31/133; A61K 31/27; A61K 31/343; A61K 31/407; A61K 31/4406; A61K 31/4409; A61K 31/496; A61K 31/4965; A61K 31/5575; A61K 31/5578; A61K 31/7036; A61K 31/7048; A61K 31/7052; A61P 31/04

(57) ABSTRACT

The invention relates to the treatment of nontuberculous mycobacterial infections. In particular, the invention relates to the use of prostanoid receptor modulators to treat nontuberculous mycobacterial infections.

### FIELD OF THE INVENTION

The invention relates to the treatment of nontuberculous mycobacterial infections. In particular, the invention relates to the use of prostanoid receptor modulators to treat nontuberculous mycobacterial infections.

### BACKGROUND OF THE INVENTION

The Mycobacterium genus encompasses both M. tuberculosis complex, the highest single infectious cause of death globally prior to COVID-19, and non-tuberculous mycobacteria (NTM). NTM are ubiquitous environmental microorganisms.

Effective human immunity against mycobacteria remains poorly understood. The live attenuated Bacillus Calmette—Guérin (BCG) vaccine derived from M. bovis induces immunity to M. tuberculosis infection, protects against severe tuberculosis (TB) infection in children, and also against NTM infections. However, in patients with a compromised immune system, BCG itself can give rise to pathological infection. Although young children are particularly susceptible to TB and have high mortality, school age children appear to be relatively protected against disease. Host inflammation and in particular eicosanoid mediators are implicated in pathogenesis in TB meningitis, but have not yet been investigated as the biological basis for effective natural human immunity against TB infection.

To-date the principle focus of Mycobacterium research has been on M. tuberculosis. However, NTM are significant pathogens in those with underlying chronic respiratory disorders or significantly immunocompromised. NTM are extremely difficult to treat clinically as they are often drug-resistant and require long courses of multidrug therapy including intravenous medication with significant side effects and toxicities and poor cure rates. NTMs are associated with significant morbidity, mortality, prolonged hospitalisation, and increased healthcare systems costs. Having an NTM infection that has not responded to treatment can affect patient prognosis so much that it can be a contraindication to lung transplant.

Therefore, there remains an unmet clinical need for safe and effective treatments for NTM infection. In particular, less toxic, shorter course and/or more effective treatments are in dire need. It is an object of the present invention to address one or more of these problems.

### SUMMARY OF THE INVENTION

The present inventors have identified that the prostacyclin pathway is implicated in NTM infections. Surprisingly, the present inventors have also demonstrated that the prostacyclin analogue Treprostinil, which is an EP₂ receptor agonist that is conventionally used to treat pulmonary hypertension, significantly increases killing of M. abscessus mycobacteria in an in vitro human monocyte macrophage cell line infection model, whereas the effect of Treprostinil on M. tuberculosis was much less significant. Of particular clinical interest, the inventors have shown in vivo that the activity of Treprostinil is host-mediated, and in particular that whilst Treprostinil has a host-directed effect against NTM, no activity was observed in a zebrafish model against M. tuberculosis-like M. marinum, allowing the potential to augment antibiotic treatment of NTM infections in patients, including those who are immunocompromised and otherwise unable to resolve NTM infection. Also of particular clinical significance, the inventors have demonstrated that the efficacy of Treprostinil is significantly greater than that of the intravenous carbapenem antibiotic imipenem that is used clinically as part of NTM treatment, and that there is a strong dose-response to Treprostinil treatment. All of these results support the clinical potential of using prostanoid receptor modulators such as Treprostinil in the treatment of NTM, where conventional treatment options are few and of limited clinical efficacy.

Therefore, the present invention provides a new means of treating NTM infections, with clinical potential as an adjunctive host-directed therapy to existing or novel antibiotic combination regimens.

Accordingly, the invention provides a prostanoid receptor modulator for use in a method of treating or preventing a nontuberculous mycobacterial infection. Said prostanoid receptor modulator may be (a) a prostanoid receptor agonist; or (b) a prostanoid receptor antagonist, preferably a prostanoid receptor agonist. Said prostanoid receptor modulator preferably binds to the EP₂ receptor. Preferably the prostanoid receptor modulator for use according to the invention is a prostanoid receptor agonist, optionally (a) a prostacyclin analogue; or (b) a non-prostanoid prostanoid receptor agonist. Said prostacyclin analogue may be a PGI₂ analogue, which may optionally be selected from Treprostinil, Iloprost, Epoprostenol, Beraprost and Cisaprost. Preferably said prostacyclin analogue may be Treprostinil. Said non-prostanoid prostanoid receptor agonist may be selected from Selexipag, MRE-269, Ralinepag (APD811), Esuberaprost and ONO-1301.

The prostanoid receptor modulator may be used in the treatment or prevention of a nontuberculous mycobacterial infection with a nontuberculous mycobacteria (NTM) preferably selected from Mycobacterium abscessus, Mycobacterium avium Complex (MAC), Mycobacterium chelonae, Mycobacterium fortuitum, Mycobacterium kansasii, Mycobacterium scrofulaceum, Mycobacterium smegmatis, Mycobacterium ulcerans, Mycobacterium xenopi and Mycobacterium bovis Bacillus Calmette-Guerin (BCG), preferably Mycobacterium abscessus, BCG and/or MAC.

According to the invention, the prostanoid receptor modulator may preferably have host-directed activity against the NTM. Said host-directed effect may be mediated by monocytes and/or macrophages.

The NTM treated according to the invention may be a drug-resistant NTM, optionally a multi-drug resistant NTM.

Treatment with a prostanoid receptor modulator of the invention may be more efficacious than treatment with antibiotic therapy. Preferably, adjunctive therapy with a prostanoid receptor modulator of the invention and antibiotic therapy/treatment may be more efficacious than therapy with (i) the antibiotic therapy/treatment alone, or (ii) the prostanoid receptor modulator alone. Said antibiotic therapy/treatment may be any conventionally indicated for NTM infection, preferably comprising a carbapenem antibiotic, more preferably imipenem.

The prostanoid receptor modulator may be for inhalative, nebulised, intravenous, subcutaneous injection or oral administration, preferably inhalation, more preferably oral inhalation.

The prostanoid receptor modulator for use according to the invention may be used in combination with a second therapy for nontuberculous mycobacterial infection. Said second therapy for nontuberculous mycobacterial infection may be an antibiotic therapy. Said antibiotic therapy may be selected from: (a) a carbapenem antibiotic; and/or (b) one or more of amikacin, azithromycin, clarithromycin, erythromycin, imipenem, isoniazid, rifampicin, ethambutol, and pyrazinamide, preferably one or more of amikacin, azithromycin, clarithromycin, erythromycin, imipenem or rifampicin.

The prostanoid receptor modulator when used in combination with a second therapy for nontuberculous mycobacterial infection according to the invention may: (a) reduce the number of agents required for the second therapy; (b) reduce the dose of agents required for the second therapy; (c) reduce the duration required for the second therapy, optionally the duration of i.v. administration of the second therapy; (d) reduce the need for requirement for intravenous administration of agents for the second therapy; (e) reduce one or more side effect associated with the second therapy; (f) reduce the duration of hospital admission; (g) improve patient quality of life, optionally as measured using the St. George Respiratory Questionnaire (SGRQ); (h) improve patient respiratory function; and/or (i) maintain a patient's eligibility for additional treatments, preferably transplant eligibility, more preferably lung transplant eligibility.

A prostanoid receptor modulator for use according to the invention may be used to treat a patient for a nontuberculous mycobacterial infection, which patient may have a pre-existing disease or disorder. Said patient may have (a) a chronic lung disease; (b) an immunodeficiency, optionally a primary immunodeficiency, or an acquired or secondary immunodeficiency; (c) an indwelling prosthetic or indwelling prosthetic material; and/or (d) has undergone open heart surgery. Said patient may have cystic fibrosis and/or bronchiectasis; and/or the nontuberculous mycobacterial infection may be an M. abscessus infection. Treatment with a prostanoid receptor modulator according to the invention may maintain a patient's eligibility for a lung transplant that would otherwise have been contraindicated. A patient to be treated may not have tuberculosis.

The invention also provides a method of treating or preventing a nontuberculous mycobacterial infection, comprising administering a therapeutically effective amount of a prostanoid receptor modulator to a patient in need thereof.

The invention further provides the use of a prostanoid receptor modulator in the manufacture of a medicament for the treatment or prevention of a nontuberculous mycobacterial infection.

The invention also provides a method of screening a patient for suitability for a treatment as according to the invention, said method comprising: (a) determining the amount of 6-keto-PGF1-α in a sample from the patient; and/or (b) determining the expression level of a prostanoid receptor in a sample from the patient; and identifying the patient as suitable for said treatment when the amount of 6-keto-PGF1-α and/or the expression level of a prostanoid receptor is above a threshold level. Said prostanoid receptor may be EP₂; and/or gene expression of PTGER2 may be determined.

The invention also provides a prostanoid receptor agonist for use in a method of treating or preventing a nontuberculous mycobacterial infection. Said prostanoid receptor agonist is preferably an EP2 receptor agonist, more preferably said prostanoid receptor agonist binds to the EP2 receptor. Preferably the prostanoid receptor agonist for use according to the invention is (a) a prostacyclin analogue; or (b) a non-prostanoid prostanoid receptor agonist. Said prostacyclin analogue (also referred to as a PGI₂ analogue) may optionally be selected from Treprostinil, Iloprost, Epoprostenol, Beraprost and Cisaprost. Preferably said prostacyclin analogue may be Treprostinil. Said non-prostanoid prostanoid receptor agonist may be selected from Selexipag, MRE-269, Ralinepag (APD811), Esuberaprost and ONO-1301.

The prostanoid receptor agonist may be used in the treatment or prevention of a nontuberculous mycobacterial infection with a nontuberculous mycobacteria (NTM) preferably selected from Mycobacterium abscessus, Mycobacterium avium Complex (MAC), Mycobacterium chelonae, Mycobacterium fortuitum, Mycobacterium kansasii, Mycobacterium scrofulaceum, Mycobacterium smegmatis, Mycobacterium ulcerans, Mycobacterium xenopi and Mycobacterium bovis Bacillus Calmette-Guerin (BCG), preferably Mycobacterium abscessus, BCG and/or MAC.

According to the invention, the prostanoid receptor agonist may preferably have host-directed activity against the NTM. Said host-directed effect may be mediated by monocytes and/or macrophages.

The NTM treated according to the invention may be a drug-resistant NTM, optionally a multi-drug resistant NTM.

Treatment with a prostanoid receptor agonist of the invention may be more efficacious than treatment with antibiotic therapy. Preferably, adjunctive therapy with a prostanoid receptor agonist of the invention and antibiotic therapy/treatment may be more efficacious than therapy with (i) the antibiotic therapy/treatment alone, or (ii) the prostanoid receptor agonist alone. Said antibiotic therapy/treatment may be any conventionally indicated for NTM infection, preferably comprising a carbapenem antibiotic, more preferably imipenem.

The prostanoid receptor agonist may be for inhalative, nebulised, intravenous, subcutaneous injection or oral administration, preferably inhalation, more preferably oral inhalation.

The prostanoid receptor agonist for use according to the invention may be used in combination with a second therapy for nontuberculous mycobacterial infection. Said second therapy for nontuberculous mycobacterial infection may be an antibiotic therapy. Said antibiotic therapy may be selected from: (a) a carbapenem antibiotic; and/or (b) one or more of amikacin, azithromycin, clarithromycin, erythromycin, imipenem, isoniazid, rifampicin, ethambutol, and pyrazinamide, preferably one or more of amikacin, azithromycin, clarithromycin, erythromycin, imipenem or rifampicin.

The prostanoid receptor agonist when used in combination with a second therapy for nontuberculous mycobacterial infection according to the invention may: (a) reduce the number of agents required for the second therapy; (b) reduce the dose of agents required for the second therapy; (c) reduce the duration required for the second therapy, optionally the duration of i.v. administration of the second therapy; (d) reduce the need for requirement for intravenous administration of agents for the second therapy; (e) reduce one or more side effect associated with the second therapy; (f) reduce the duration of hospital admission; (g) improve patient quality of life, optionally as measured using the St. George Respiratory Questionnaire (SGRQ); (h) improve patient respiratory function; and/or (i) maintain a patient's eligibility for additional treatments, preferably transplant eligibility, more preferably lung transplant eligibility.

A prostanoid receptor agonist for use according to the invention may be used to treat a patient for a nontuberculous mycobacterial infection, which patient may have a pre-existing disease or disorder. Said patient may have (a) a chronic lung disease; (b) an immunodeficiency, optionally a primary immunodeficiency, or an acquired or secondary immunodeficiency; (c) an indwelling prosthetic or indwelling prosthetic material; and/or (d) has undergone open heart surgery. Said patient may have cystic fibrosis and/or bronchiectasis; and/or the nontuberculous mycobacterial infection may be an M. abscessus infection. Treatment with a prostanoid receptor agonist according to the invention may maintain a patient's eligibility for a lung transplant that would otherwise have been contraindicated. A patient to be treated may not have tuberculosis.

The invention also provides a method of treating or preventing a nontuberculous mycobacterial infection, comprising administering a therapeutically effective amount of a prostanoid receptor agonist to a patient in need thereof.

The invention further provides the use of a prostanoid receptor agonist in the manufacture of a medicament for the treatment or prevention of a nontuberculous mycobacterial infection.

### BRIEF DESCRIPTION OF THE DRAWINGS

FIG. 1: 6-keto-PGF1-α levels following BCG infection of whole blood samples from Gambian children with M. tuberculosis exposure but negative tuberculin skin test (High Exposure Uninfected, HEU) and matched children with positive tuberculin skin test and TB infection (High Exposure Infected, HEI) at baseline (A) and at 24 hours (B).

FIG. 2: Levels of PTGER2 mRNA, a prostacyclin agonist receptor, in unstimulated whole blood samples from HEU and HEI children.

FIG. 3: PTGER2 and PTGS2 mRNA expression following M. tuberculosis infection (U=unstimulated, S=stimulated) of primary monocytes from Vietnamese adults with latent TB (LTBI), compared to those with pulmonary (PTB) or meningeal TB (TBM).

FIG. 4: Addition of Treprostinil at 400 nM (square) and 1 μM (triangle) concentrations to THP1 cells infected by M. abscessus (A) and M. tuberculosis (B) against an untreated control (circle). (C) Addition of Treprostinil or Rifampicin to zebrafish infected by M. marinum.

FIG. 5: (A) Treatment with Treprostinil at 50 μM is more effective at killing M. abscessus than treatment with imipenem. (B) Treatment with Treprostinil elicited a dose-response in THP1 killing of M. abscessus.

FIG. 6: Addition of 50 μM (square) of Treprostinil to M. abscessus in liquid culture medium showed no significant antibiotic effect on mycobacterial growth compared with a negative control (circle), in contrast to the positive control, imipenem (triangle).

FIG. 7: CRISPR KO of zebrafish EP2a completely abrogated Treprostinil rescue (A) graph quantifying and (B) representative images showing effect of Treprostinil on M. abscessus in control and FO CRISPR EP2a KO zebrafish.

### DETAILED DESCRIPTION OF THE INVENTION

### Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure. The meaning and scope of the terms should be clear; however, in the event of any latent ambiguity, definitions provided herein take precedent over any dictionary or extrinsic definition.

It should be understood that this invention is not limited to the particular methodology, protocols, and reagents, etc., described herein and as such can vary. In particular, any methods and materials similar or equivalent to those described herein can be used in the practice or testing of embodiments of this disclosure.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

The headings provided herein are not limitations of the various aspects or embodiments of this disclosure.

As used herein, the term “capable of” when used with a verb, encompasses or means the action of the corresponding verb. For example, “capable of interacting” also means interacting, “capable of cleaving” also means cleaves, “capable of binding” also means binds and “capable of specifically targeting . . . ” also means specifically targets.

Numeric ranges are inclusive of the numbers defining the range. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

The terms “increased”, “increase”, “enhance”, or “activate” are all Used herein to mean an increase by a statistically significant amount. The terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. By way of non-limiting example, in the context of mycobacterial growth, an “increase” is an observable or statistically significant increase in such level.

The terms “decrease”, “reduced”, “reduction”, or “inhibit” are all used herein to mean a decrease by a statistically significant amount. The terms “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” encompasses a complete inhibition or reduction as compared to a reference level. “Complete inhibition” is a 100% inhibition (i.e. abrogation) as compared to a reference level.

An “analogue” of a chemical structure, as the term is used herein, refers to a chemical structure that preserves substantial similarity with the parent structure, although it may not be readily derived synthetically from the parent structure.

A related chemical structure that is readily derived synthetically from a parent chemical structure is referred to as a “derivative”.

A molecule that mimics the effect (e.g. function/activity) of parent molecule, such as a prostacyclin, may be referred to herein as a mimetic. A mimetic may or may not be structurally related to the parent molecule.

The term “chemically feasible” means a bonding arrangement or a compound where the generally understood rules of organic structure are not violated; for example a structure within a definition of a claim that would contain in certain situations a pentavalent carbon atom that would not exist in nature would be understood to not be within the claim. The structures disclosed herein, in all of their embodiments are intended to include only “chemically feasible” structures, and any recited structures that are not chemically feasible, for example in a structure shown with variable atoms or groups, are not intended to be disclosed herein and do not form part of the present invention.

A “prodrug” as is well known in the art is a substance that can be administered to a patient where the substance is converted in vivo by the action of biochemicals within the patient's body, such as enzymes, to the active pharmaceutical ingredient. Examples of prodrugs include esters of carboxylic acid groups, which can be hydrolyzed by endogenous esterases as are found in the bloodstream of humans and other mammals. Conventional procedures for the selection and preparation of suitable prodrug derivatives are described, for example, in “Design of Prodrugs”, ed. H. Bundgaard, Elsevier, 1985.

As used herein, the terms “host-directed therapeutic” and “host targeted therapeutic” refer to therapeutics which act via a host-mediated response to pathogens (in this invention NTMs), rather than acting directly on said pathogen.

It must be noted that as used herein and in the appended claims, the singular forms “a”, “an”, and “the” include plural referents unless the context clearly dictates otherwise. Thus, for example, reference to “a prostanoid receptor modulator” includes a plurality of such agents and reference to “the prostanoid receptor modulator” includes reference to one or more prostanoid receptor modulators and equivalents thereof known to those skilled in the art, and so forth. Furthermore, the use of the term “including”, as well as other forms, such as “includes” and “included”, is not limiting.

As used herein the terms “adjunctive therapy”, “adjuvant therapy”, “adjunct therapy”, and “augmentation therapy” describe a therapy that is given in addition to the primary or initial therapy to maximise its effectiveness.

“About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (±) 5%, preferably ±4%, ±3%, ±2%, ±1%, ±0.5%, ±0.1%, of the numerical value of the number with which it is being used.

The term “consisting of” refers to compositions, methods, and respective components thereof as described herein, which are exclusive of any element not recited in that description of the invention.

As used herein the term “consisting essentially of” refers to those elements required for a given invention. The term permits the presence of elements that do not materially affect the basic and novel or functional characteristic(s) of that invention (i.e. inactive or non-immunogenic ingredients).

Embodiments described herein as “comprising” one or more features may also be considered as disclosure of the corresponding embodiments “consisting of” and/or “consisting essentially of” such features.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

The terms “individual”, “subject”, and “patient”, are used interchangeably herein to refer to a mammalian subject for whom diagnosis, prognosis, disease monitoring, treatment, therapy, and/or therapy optimisation is desired. The mammal can be (without limitation) a human, non-human primate, mouse, rat, dog, cat, horse, or cow. In a preferred embodiment, the individual, subject, or patient is a human. An “individual” may be an adult, juvenile or infant. An “individual” may be male or female.

A “subject in need” of treatment for a particular condition can be an individual having that condition, diagnosed as having that condition, or at risk of developing that condition.

A subject can be one who has been previously diagnosed with or identified as suffering from or having a condition in need of treatment or one or more complications related to such a condition, and optionally, have already undergone treatment for a condition as defined herein or the one or more complications related to said condition. Alternatively, a subject can also be one who has not been previously diagnosed as having a condition as defined herein or one or more complications related to said condition. For example, an individual can be one who exhibits one or more risk factors for a condition, or one or more complications related to said condition or a subject who does not exhibit risk factors.

As used herein, the term “healthy individual” refers to an individual or group of individuals who are in a healthy state, e.g. individuals who have not shown any symptoms of the disease, have not been diagnosed with the disease and/or are not likely to develop the disease (e.g. NTM infection or any other disease described herein). Preferably said healthy individual(s) is not on medication affecting NTM infection and has not been diagnosed with any other disease. The one or more healthy individuals may have a similar sex, age, and/or body mass index (BMI) as compared with the test individual. Application of standard statistical methods used in medicine permits determination of normal levels of expression in healthy individuals, and significant deviations from such normal levels.

The effect of a treatment of the invention may be compared with a suitable control or reference population, such as a healthy individual, or an individual with NTM (typically the same individual) prior to treatment.

Herein the terms “control” and “reference population” are used interchangeably.

The term “pharmaceutically acceptable” as used herein means approved by a regulatory agency of the Federal or a state government, or listed in the U.S. Pharmacopeia, European Pharmacopeia or other generally recognized pharmacopeia.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

The publications discussed herein are provided solely for their disclosure prior to the filing date of the present application. Nothing herein is to be construed as an admission that such publications constitute prior art to the claims appended hereto. All references cited in this specification are herewith incorporated by reference with respect to their entire disclosure content and the disclosure content specifically mentioned in this specification.

Disclosure related to the various methods of the invention are intended to be applied equally to other methods, therapeutic uses or methods, and vice versa.

### Prostanoid Receptors

As exemplified herein, the present inventors have found that the prostacyclin pathway is implicated in NTM infections. In particular, the inventors have shown that a prostacyclin analogue, Treprostinil, acts on the EP₂ receptor, which is part of the prostacylin pathway (amongst other pathways, including PGE₂). Further, the inventors have shown that targeting the prostacyclin pathway can increase killing of mycobacteria. According to the invention, the prostacyclin pathway can be targeted using modulators of prostanoid receptors.

Accordingly, the invention provides prostanoid receptor modulators for use in methods of treating or preventing NTM infection. Preferably, the invention provides prostanoid receptor agonists for use in methods of treating or preventing NTM infection. Preferably, the prostanoid receptor agonist is an EP₂ receptor agonist. Typically such EP₂ receptor agonists bind to the EP₂ receptor. In a particularly preferred embodiment, the prostanoid receptor agonist is a prostacyclin analogue and binds the EP₂ receptor.

Prostanoids are a subclass of eicosanoids that are produced by a pathway involving the conversion of arachidonic acid by cyclooxygenase isoenzymes (COX-1 and COX-2) to prostaglandin H₂ (PGH₂). PGH₂ is the common precursor for five principal bioactive derivatives, referred to as prostanoids. These prostanoids are thromboxane (TXA₂), prostaglandin F_{2α} (PGF_{2α}), prostaglandin E₂ (PGE₂), prostacyclin (PGI₂) and prostaglandin D₂ (PGD₂).

Prostanoids act by binding to and activating specific membrane bound G-protein-coupled receptors (GPCR). TXA₂ binds to the TP receptor, PGF_{2α} binds to the FP receptor, PGE₂ binds to receptors EP₁, EP₂ and EP₃. PGD₂ binds to DP₁. PGI₂ binds to EP₂, EP₁, EP₃, DP₁ and IP, as well as to peroxisome proliferator-activated receptors (PPARs).

EP₁, EP₃, FP and TP receptors are contractile receptors coupled to Gq and Gi that either elevate intracellular Ca²⁺ and/or reduce cAMP, respectively. The EP₂, IP and DP₁ receptors are vasorelaxant receptors coupled to Gs that will activate adenylate cyclase and elevate intracellular cAMP.

As demonstrated herein, prostanoid receptors, particularly EP₂, are implicated in NTM infection. Further, the inventors have shown that targeting prostanoid receptors such as EP₂ using modulators, particularly prostanoid receptor agonists such as prostacyclin analogues, for these prostanoid receptors increases killing in a THP1 infection model and in a host-directed manner in zebrafish studies with M. abscessus, which supports the potential of prostanoid receptor modulators, particularly prostanoid receptor agonists such as prostacyclin analogues, to increase the NTM killing activity of macrophages. As such, the invention relates to the use of prostanoid receptor modulators for the treatment and/or prevention of NTM infection. Preferably, the invention relates to prostanoid receptor agonists for use in methods of treating or preventing NTM infection.

A prostanoid receptor modulator of the invention, preferably a prostanoid receptor agonist, may specifically modulate one or more prostanoid receptor.

A prostanoid receptor modulator according to the invention, preferably a prostanoid receptor agonist, may selectively modulate one or more prostanoid receptor (also referred to interchangeably herein as specifically modulating one or more prostanoid receptor). This is typically the case for agents which directly modulate a prostanoid receptor. For such direct prostanoid receptor modulators, selectivity may mean that the modulator binds selectively (also referred to interchangeably herein as specifically) to one or more prostanoid receptor. By “binds selectively”, it will be understood that said prostanoid receptor modulator binds to one or more prostanoid receptor, with no significant cross-reactivity to any other molecules. Cross-reactivity may be assessed by any suitable method. By way of non-limiting example, cross-reactivity of a prostanoid receptor modulator with a molecule other than said one or more prostanoid receptor may be considered significant if the agent binds to the other molecule at least 5%, 10%, 15%, 20%, 25%, 30%, 35%, 40%, 45%, 50%, 55%, 60%, 65%, 70%, 75%, 80%, 85%, 90% or 100% as strongly as it binds to the one or more prostanoid receptor. A prostanoid receptor modulator that binds selectively to one or more prostanoid receptor may bind to another molecule at less than 90%, 85%, 80%, 75%, 70%, 65%, 60%, 55%, 50%, 45%, 40%, 35%, 30%, 25% or 20% the strength that it binds to said one or more prostanoid receptor. Preferably, the prostanoid receptor modulator binds to the other molecule at less than 20%, less than 15%, less than 10% or less than 5%, less than 2% or less than 1% the strength that it binds to said one or more prostanoid receptor. By way of non-limiting example, a prostanoid receptor modulator of the invention may have no significant cross-reactivity with a phosphodiesterase (PDE), sodium channel and/or calcium channel.

A prostanoid receptor modulator of the invention, preferably a prostanoid receptor agonist, may have off-target effects. An off-target effect is activity against a target other than said prostanoid receptor. Typically compounds with off-target effects are encompassed by the present invention if the activity against the non-prostanoid receptor target is not significant compared with the activity against the prostanoid receptor. Whether an off-target effect is significant may depend on the intended use of the compound. As a non-limiting example, a compound which may exert an off-target effect on the central nervous system may or may not be significant (depending on the magnitude of the off-target effect) for an in vivo therapeutic indication as disclosed herein. The presence and magnitude of any potential off target effects can be readily assessed using standard methods known in the art.

A prostanoid receptor modulator of the invention, preferably a prostanoid receptor agonist of the invention, may modulate one or more prostanoid receptor, such as any 2, any 3, any 4 or more prostanoid receptors. Thus, a prostanoid receptor modulator may modulate one or more of EP₂, EP₁, EP₃, FP, TP, IP and/or DP₁, or any combination thereof. By way of non-limiting example, a prostanoid receptor modulator, preferably a prostanoid receptor agonist, may modulate both EP₂ and IP.

Alternatively, a prostanoid receptor modulator of the invention, preferably a prostanoid receptor agonist, may modulate a single prostanoid receptor. Thus, a prostanoid receptor modulator may modulate one of EP₂, EP₁, EP₃, FP, TP, IP, DP₁ or a PPARs. Preferably, a prostanoid receptor modulator, preferably a prostanoid receptor agonist, may modulate EP₂. More preferably, said prostanoid receptor agonist is a prostacyclin analogue that may modulate EP₂.

An exemplary EP₁ receptor sequence is provided by UniProt Accession No. P34995 (accessed 5 Jul. 2022, sequence version 3). An exemplary EP₂ receptor sequence is provided by UniProt Accession No. P43116 (accessed 5 Jul. 2022, sequence version 2). An exemplary EP₃ receptor sequence is provided by UniProt Accession No. P43115 (accessed 5 Jul. 2022, sequence version 1). An exemplary IP receptor sequence is provided by UniProt Accession No. P43119 (accessed 5 Jul. 2022, sequence version 1). An exemplary TP receptor sequence is provided by UniProt Accession No. P21731 (accessed 5 Jul. 2022, sequence version 3). An exemplary FP receptor sequence is provided by UniProt Accession No. P43088 (accessed 5 Jul. 2022, sequence version 1). An exemplary DP₁ receptor sequence is provided by UniProt Accession No. Q13258 (accessed 5 Jul. 2022, sequence version 2).

### Prostanoid Receptor Modulators

The invention relates to both direct and indirect modulation of prostanoid receptors. The term “modulation of a prostanoid receptor” encompasses both up-and down-regulation of (i.e. increasing and decreasing as defined herein) both prostanoid receptor activity and/or expression. Typically the invention relates to modulation of prostanoid receptor activity, i.e. signalling through the prostanoid receptor.

Unless explicitly stated, references herein to modulation of prostanoid receptor encompass both direct and indirect modulation of prostanoid receptor. Agents which modulate prostanoid receptor (either directly or indirectly) are referred to herein as “prostanoid receptor modulators”. Preferably, the invention relates to direct modulation of prostanoid receptor, wherein a prostanoid receptor modulator binds to prostanoid receptor and directly modulates its activity.

“Direct modulation of prostanoid receptors” as used herein means modulation of the activity and/or expression of prostanoid receptors, i.e. without any intermediary step. By way of non-limiting example, direct up-regulation may be achieved, for example, by use of a prostanoid receptor agonist, such as a prostacyclin analogue. By way of a further non-limiting example, direct down-regulation may be achieved, for example, by use of a prostanoid receptor antagonist. Preferably, prostanoid receptor modulators of the invention are used to directly modulate the activity of one or more prostanoid receptors activity. Preferably said prostanoid receptor modulators are prostanoid receptor agonists such as a prostacyclin analogue.

“Indirect modulation of prostanoid receptors” as used herein means modulation of the activity and/or expression of prostanoid receptors indirectly, i.e. through the modulation or delivery of genes/enzymes up-or down-stream of prostanoid receptors and/or through the generation or delivery of intermediaries which directly modulate said prostanoid receptors. Indirect modulation may be elicited by upregulating the expression of an enzyme which generates an endogenous direct modulator of said prostanoid receptors.

Prostanoid receptor activity may be measured relative to a control. Thus, the activity of a prostanoid receptor treated according to the invention may be compared with the activity of said prostanoid receptor in a control. Activity may be quantified in any appropriate terms, for example downstream effects such as changes in intracellular Ca²⁺ and/or cAMP concentrations, or in terms of NTM killing activity as defined herein. Any appropriate technique or method may be used for quantifying prostanoid receptor activity. Suitable techniques are known in the art, for example calcium flux assays, Fura-2 or Fluo-4 assay to measure intracellular Ca²⁺, or assays to determine growth and/or killing activity of cells against NTMs.

Expression may be quantified in terms of gene and/or protein expression, and may be compared with the expression of a control (e.g. housekeeping gene or protein). As a non-limiting example, in the context of prostanoid receptor expression, the actual amount of a prostanoid receptors gene, mRNA transcript and/or protein, such as the mass, molar amount, concentration or molarity of a prostanoid receptor gene, mRNA transcript and/or protein, or the number of mRNA molecules per cell in a sample obtained from an individual treated according to the invention and the control may be assessed, and compared with the corresponding value from the control. Alternatively, the expression of a prostanoid receptors gene and/or protein in a sample obtained from an individual treated according to the invention may be compared with that of the control without quantifying the mass, molar amount, concentration or molarity of the one or more gene and/or protein.

Typically, the control is an equivalent sample in which no modulation of prostanoid receptor expression or activity has been effected. As a non-limiting example, in the case where an individual is treated with an prostanoid receptor modulator that modulates prostanoid receptor expression, a suitable control would be a different individual to which the prostanoid receptor modulator has not been administered or the same individual prior to administration of the modulator. Conventional methods for the assessment of gene and/or protein expression are well known in the art and include RT-qPCR, ELISA, DNA microarray, RNA Seq, serial analysis of gene expression (SAGE) and western blotting. As a further non-limiting example, in the case where an individual is treated with a prostanoid receptor modulator that modulates prostanoid receptor activity, a suitable control would be a different individual to which the modulator has not been administered or the same individual prior to administration of the modulator.

In the context of the present invention, when referring to (direct or indirect) modulation of prostanoid receptor activity and/or expression, the degree of modulation (increase or decrease of prostanoid receptor activity and/or expression) may be as defined above.

Any suitable agent which modulates prostanoid receptor may be used according to the present invention. Preferably prostacyclin analogues are used. Non-limiting examples of suitable prostanoid receptor modulators include small molecules, antibodies and antigen-binding fragments thereof, peptides and peptidomimetics, nucleic acids and aptamers, as described herein.

Typically agents which directly modulate prostanoid receptors are used. Preferably said modulators bind to one or more prostanoid receptors. An agent which directly modulates one or more prostanoid receptor, may be selected from a small molecule, a nucleic acid (for example, an siRNA, shRNA, or antisense oligonucleotide), antibody or antigen-binding fragment, or an aptamer. Other non-limiting examples of agents include cell therapy agents or immunotherapeutics and gene therapies. The term “immunotherapeutic” encompasses agents such as recombinant T-cells and/or bacteriophages, which may comprise a cell-targeting moiety (e.g. a TCR or CAR in the case of a recombinant T cell) which binds to a prostanoid receptor. Also encompassed are T cells, both those selected from a donor population or recombinant, that target NTM and express EP₂ receptors, such that combination treatment with a prostanoid receptor modulator such as Treprostinil may augment their anti-NTM activity. Preferably a prostanoid receptor modulator which directly modulates one or more prostanoid receptor is a binding member and/or a small molecule, even more preferably both.

An agent which indirectly modulates one or more prostanoid receptor may be selected from a small molecule, a nucleic acid (for example, an siRNA, shRNA, or antisense oligonucleotide), antibody or antigen-binding fragment, or an aptamer.

The prostanoid receptor modulators of the present invention herein may be small molecules. As defined herein, small molecules are low molecular weight compounds, typically organic compounds. Typically, a small molecule has a maximum molecule weight of 900 Da, allowing for rapid diffusion across cell membranes. In some embodiments, the maximum molecular weight of a small molecule is 500 Da. Typically a small molecule has a size in the order of 1 nm.

As described in more detail below, the term prostanoid receptor modulator encompasses both prostanoid receptor agonists and antagonists. The selection of an agonist or antagonist may depend in the specific prostanoid receptor, and/or the NTM to be treated. By way of non-limiting example, as demonstrated herein, increased expression levels of some prostanoid receptors, such as EP₂, may be associated with decreased susceptibility to NTM infection. As such, modulators of these prostanoid receptors according to the invention may preferably be prostanoid receptor agonists. Preferably, said prostanoid receptor agonists are prostacyclin analogues.

### Prostanoid Receptor Agonists

The invention relates to the use of prostanoid receptor agonists to increase prostanoid receptor activity. In the context of the present invention a prostanoid receptor agonist is any compound which stimulates, increases or promotes the action of said prostanoid receptor.

Prostanoid receptor agonists may act by any suitable mechanism, which typically involves binding of the agonist to the prostanoid receptor (i.e. as a form of direct modulation as described herein).

A prostanoid receptor agonist may have any feasible chemical structure provided that it increases prostanoid receptor activity. In some preferred embodiments, a prostanoid receptor agonist may be a small molecule. As used herein, the term prostanoid receptor agonists includes both prostacyclin analogues, derivatives and mimetics, as well as non-prostanoid prostanoid receptor agonists.

In some preferred instances, the prostanoid receptor agonist may be an agonist of the EP₂ receptor. Non-limiting examples of EP₂ receptor agonists include Sulprostone, Dinoprostone, Carboprost, Carboprost tromethamine, omidenepag isopropyl, misoprostol, alprostadil, JVGL-1, aganepag isopropyl, PGN-9856, R-111, R-65, R-99, simenepag isopropyl, ACP-009, Evatanepag, ONO-8055, SAR-366234, or taprenepag isopropyl. Further non-limiting examples of EP₂ receptor agonists include Sulprostone, Dinoprostone, Carboprost, Carboprost tromethamine, omidenepag isopropyl, misoprostol, alprostadil, JVGL-1, PGN-9856, Evatanepag, ONO-8055, SAR-366234, or taprenepag isopropyl. Preferably, EP₂ receptor agonists may be selected from Sulprostone, Dinoprostone, Carboprost, Carboprost tromethamine, omidenepag isopropyl, misoprostol, alprostadil, JVGL-1, PGN-9856, Evatanepag, ONO-8055, SAR-366234, and taprenepag isopropyl. More preferably, EP₂ receptor agonists may be selected from Sulprostone, Dinoprostone, Carboprost, Carboprost tromethamine, omidenepag isopropyl, misoprostol, alprostadil, JVGL-1, Evatanepag, and taprenepag isopropyl.

### Prostacyclin Analogues, Derivatives and Mimetics

A prostanoid receptor modulator, preferably a prostanoid receptor agonist, may have structural similarity with a prostanoid ligand for said prostanoid receptor. Thus, a prostanoid receptor modulator, particularly a prostanoid receptor agonist, may be a prostacyclin analogue, derivative and/or mimetic. In preferred instances, the prostanoid receptor agonist is a prostacyclin analogue. In more preferred instances, said prostacyclin analogue binds to the EP₂ receptor.

A prostacyclin analogue refers to a chemical structure that preserves substantial similarity with the parent prostacyclin structure, although it may not be readily derived synthetically from the parent prostacyclin structure.

A prostacyclin derivative is a chemical structure that is readily derived synthetically from a parent prostacyclin chemical structure.

A prostacyclin mimetic is a molecule that mimics the effect (e.g. activity/function) of the prostacyclin of interest, but may or may not be structurally related.

Thus, in some instances, the terms prostacyclin analogue, prostacyclin derivative and prostacyclin mimetic may be used interchangeably.

The invention relates to the use of prostacyclin analogues, derivatives and/or mimetics to treat and/or prevent NTM infection.

Non-limiting examples of prostacyclin analogues which may be used according to the present invention include Treprostinil, Iloprost, Epoprostenol, Beraprost and Cisaprost. In some particularly preferred embodiments, the invention relates to the use of Treprostinil to treat and/or prevent NTM infection.

### Non-Prostanoid Prostanoid Receptor Modulators

A prostanoid receptor modulator (agonist or antagonist, preferably an agonist) may be structurally distinct from a prostanoid ligand for said prostanoid receptor. Thus, a prostanoid receptor modulator (agonist or antagonist) may be a non-prostanoid prostanoid receptor modulator, such as a non-prostanoid prostanoid receptor agonist. Preferably said non-prostanoid prostanoid receptor modulator is a non-prostanoid prostanoid receptor agonist. Non-prostanoid prostanoid receptor agonists of the invention are not structurally related to prostacyclin or prostacyclin analogues, derivatives or mimetics.

The invention relates to the use of non-prostanoid prostanoid receptor modulators, particularly non-prostanoid prostanoid receptor agonists, to treat and/or prevent NTM infection.

Non-limiting examples of non-prostanoid prostanoid receptor agonists include Selexipag, MRE-269, Ralinepag (APD811), Esuberaprost and ONO-1301.

### Prostanoid Receptor Antagonists

In some embodiments, the invention relates to the use of prostanoid receptor antagonists to decrease prostanoid receptor activity. In the context of the present invention a prostanoid receptor antagonist is any compound which inhibits, decreases, suppresses or ablates the action of said prostanoid receptor, whether in part or completely.

Prostanoid receptor antagonists may act by any suitable mechanism, and preferably involves binding of the antagonist to the prostanoid receptor (i.e. as a form of direct modulation as described herein).

A prostanoid receptor antagonist may have any feasible chemical structure provided that it decreases prostanoid receptor activity. In some preferred embodiments, a prostanoid receptor antagonist may be a small molecule.

Non-limiting examples of prostanoid receptor antagonists which may be used according to the present invention include PF-04418948 and RO1138452.

### Nontuberculous Mycobacteria

The invention relates to the treatment and prevention of mycobacteria that are non-obligate human pathogens. In particular, the invention is concerned with the treatment and prevention of infections by mycobacteria which lack the virulence mechanisms of the M. tuberculosis complex (MTB complex). These mycobacteria include Mycobacterium bovis Bacillus Calmette-Guerin (BCG), as this is a form of M. bovis which has been attenuated to lack the virulence mechanisms of the MTB complex. These mycobacteria are typically only capable of causing pathology in patients with underlying conditions, particularly immunocompromised patients, and/or patients with pre-existing lung damage, although in a minority of patients these mycobacteria may cause pathology in the absence of any underlying condition. As described and exemplified herein, the invention provides prostanoid receptor modulators, preferably prostanoid receptor agonists, for use in a method of treating or preventing such mycobacterial infections, wherein the prostanoid receptor modulator, preferably the prostanoid receptor agonist, has a host-directed effect. Without being bound by theory, the present invention allows for host-directed therapy of these mycobacteria in patients whose own immune system is unable to control these mycobacterial infections. Thus, the technical effect provided by the present invention identifies a new clinical situation, with the potential for a new cohort of patients to be treated.

Thus, the invention relates to the treatment and prevention of nontuberculous mycobacteria (NTM) infection. NTM are mycobacteria other than M. tuberculosis (the cause of tuberculosis) and M. leprae (the cause of leprosy). NTM are also known in the art as atypical mycobacteria, mycobacteria other than tuberculosis (MOTT), or environmental mycobacteria.

NTM are environmental bacteria found in soil, dust, and natural/municipal water sources. NTM can form biofilms, making them difficult to eliminate in moist environments where these form, such as in catheters, air-conditioning and plumbing.

NMT are opportunistic pathogens. As such, individuals with underlying and/or chronic lung disease such as bronchiectasis, cystic fibrosis, chronic obstructive pulmonary disease (COPD) and pulmonary fibrosis are at increased risk. Similarly, individuals with depressed immune systems, whether because of a primary immunodeficiency, or an acquired/secondary immunodeficiency are also particularly susceptible to NTM infection.

There are more than 180 recognized species of NTM. Any NTM species may be treated according to the present invention. The main NTM known to cause infections in humans are Mycobacterium abscessus complex (also referred to as M. abscessus herein), Mycobacterium avium Complex (MAC), Mycobacterium chelonae, Mycobacterium mucogenicum, Mycobacterium fortuitum, Mycobacterium kansasii, Mycobacterium chimaera, Mycobacterium scrofulaceum, Mycobacterium smegmatis, Mycobacterium ulcerans, Mycobacterium malmoense, Mycobacterium haemophilum, Mycobacterium genavense, Mycobacterium szulgai, and Mycobacterium xenopi. Therefore, the invention may relate to the treatment and/or prevention of infection by one or more of M. abscessus, M. avium Complex (MAC), M. chelonae, M. mucogenicum, M. fortuitum, M. kansasii, M. chimaera, M. scrofulaceum, M. smegmatis, M. ulcerans, M. malmoense, M. haemophilusenavense, M. szulgai, and M. xenopi. Treatment and/or prevention of one or more of M. abscessus, M. avium Complex (MAC), M. chelonae, M. mucogenicum, M. fortuitum, M. kansasii, M. chimaera, M. scrofulaceum, M. smegmatis, M. ulcerans, M. malmoense, M. haemophilusenavense, M. szulgai, and M. xenopi is preferred, particularly treatment of M. abscessus.

As used herein, the term NTM also encompasses BCG, as for the reasons discussed above it is a non-obligate human pathogen lacking the virulence mechanisms of the MTB complex, and which is capable of causing pathology only in immunocompromised patients, and/or patients with lung damage. Thus, the term NTM as used herein may also refer to M. abscessus, MAC, M. chelonae, M. mucogenicum, M. fortuitum, M. kansasii, M. chimaera, M. scrofulaceum, M. smegmatis, M. ulcerans, M. malmoense, M. haemophilusenavense, M. szulgai, and M. xenopi and/or BCG. Therefore, the invention may relate to the treatment and/or prevention of infection by one or more of M. abscessus, MAC, M. chelonae, M. mucogenicum, M. fortuitum, M. kansasii, M. chimaera, M. scrofulaceum, M. smegmatis, M. ulcerans, M. malmoense, M. haemophilusenavense, M. szulgai, M. xenopi and BCG. Treatment and/or prevention of one or more of M. abscessus, MAC, M. chelonae, M. mucogenicum, M. fortuitum, M. kansasii, M. chimaera, M. scrofulaceum, M. smegmatis, M. ulcerans, M. malmoense, M. haemophilusenavense, M. szulgai, and M. xenopi and BCG is preferred, particularly treatment of M. abscessus and/or BCG.

NTM can be divided into two groups based on how long they take to grow in a culture. Rapid-growing NTM include M. abscessus, M. chelonae, M. fortuitum and M. mucogenicum. Rapid-growing NTM typically require less than or equal to 7 days to produce mature colonies on solid media from a dilute inoculum. Slow-growing NTM include M. avium Complex, M. chimaera, M. kansasii, M. xenopi, M. malmoense, M. haemophilusenavense, M. szulgai and M. scrofulaceum. Slow-growing NTM typically require more than 7 days to produce mature colonies on solid media from a dilute inoculum. Further description and classification of NTMs is provided in Forbes et al. (Clin. Microbiol. Rev. (2018) 31(2):e00038-17), which is herein incorporated by reference in its entirety.

According to the invention, M. marinum does not fall within the definition of a NTM. This is because M. marinum is used as a “M. tuberculosis-like” pathogen in the context of the zebrafish host, as the basis of the zebrafish model of tuberculosis. Accordingly, as described and exemplified herein, the invention relates to the treatment and prevention of NTM infection and expressly excludes the treatment and/or prevention of M. marinum infection.

NTMs can cause infections in a wide variety of body sites, most commonly the lungs and respiratory tract. Other common sites of infection include the skin and soft tissue, device associated infections, lymph nodes, blood and immunoprivileged sites. Infection may follow surgery, trauma, or other interventions, such as injection of medications, or following central line insertion.

Symptoms can be vague and nonspecific, including fever, weight loss, night sweats, decreased appetite, loss of energy, cough, shortness of breath, blood in the sputum, and rashes.

### Drug Delivery Systems

A prostanoid receptor modulator of the invention, preferably a prostanoid receptor agonist, may be delivered by means of a drug delivery system. Drug delivery systems may be used to increase delivery of a prostanoid receptor modulator of the invention; increase uptake of a prostanoid receptor modulator of the invention by a target cell or tissue; and/or to increase the efficacy of a prostanoid receptor modulator of the invention.

Any appropriate drug delivery system may be used to deliver a prostanoid receptor modulator, preferably a prostanoid receptor agonist, of the invention. Conventional drug delivery systems are known in the art. By way of non-limiting example, appropriate drug delivery systems include liposomes, immunoliposomes, nanoparticles and conjugates. Thus, it would be routine for one of skill in the art to select a suitable drug delivery system. Liposome drug delivery systems are referred to interchangeably herein as liposome-based drug delivery systems.

The skilled person would understand that the choice of drug delivery system may depend on the particular indication and/or tissue to be treated.

As discussed herein, the present invention relates to the treatment and/or prevention of NTM infection. Therefore, drug delivery systems (e.g. liposomes or nanoparticles) specifically adapted for cells of the lungs and/or respiratory tract, may be used according to the invention. For example, drug delivery systems which specifically or preferentially target airway epithelial and/or endothelial cells, or cell types, such as basal cells and submucosal gland duct cells in the upper airways, club cells and neuroendocrine cells in the bronchiolar airways, bronchioalveolar stem cells in the terminal bronchioles and type II pneumocytes in the alveoli, may be used according to the invention. Such liposome drug delivery systems may further be conjugated to antibodies, or antigen binding fragments thereof, which target specific surface markers for the desired target cell type. Typically, the drug delivery systems will have an average size of between 1 to 5 μm, preferably 1.5 to 2 μm.

### Therapeutic Indications

The prostanoid receptor modulators (preferably agonists), compositions and drug delivery systems as described herein are useful in the treatment of NTM infection. NTMs are described herein.

NTM are significant pathogens, especially in those with underlying chronic respiratory disorders or significant immunocompromise, although they can cause pathology in otherwise healthy individuals, or patients who have undergone open heart surgery. NTM infections represent a significant secondary pathology in patients with depressed immune systems. Furthermore, NTM are often intrinsically drug-resistant, making them difficult to treat. Standard treatment regimens for NTM infections typically involve multidrug therapy including intravenous medication with significant side effects and toxicities and poor cure rates. Furthermore, conventional treatment regimens often involve long treatment courses, typically weeks to months in length. Typically a course of treatment may comprise an initial treatment phase of 3-12 weeks followed by maintenance therapy for up to 12 months (or until patient samples are free of NTMs). As such, NTM infections are associated with significant morbidity, mortality, prolonged hospitalisation, and increased healthcare systems costs. Having an NTM infection can affect patient prognosis so much that it can be a contraindication to lung transplant.

As demonstrated herein, M. abscessus can be treated effectively with a prostanoid receptor modulator such as Treprostinil. Although NTMs differ in some microbiological characteristics, the clinical presentations of NTM infections are common across NTMs, and the host inflammatory response to all involves prostanoids. Due to this common mechanism the prostanoid receptor modulators (preferably agonists), compositions and drug delivery systems of the invention are useful in treating any NTM infection.

Accordingly, the present invention relates to the treatment and/or prevention of NTM infection. The invention particularly relates to the treatment and/or prevention of infection by one or more species of NTM. Preferably the invention relates to the treatment of M. abscessus infection.

Without being bound by theory, it is believed that prostanoid receptor modulators, particularly prostanoid receptor agonists, used according to the present invention act via host directed mechanisms. Thus, it is believed that prostanoid receptor modulators such as prostanoid receptor agonists, exert their therapeutic effect via acting on cells of a patient to be treated, rather than by exerting a direct effect against NTM cells. In other words, prostanoid receptor modulators, particularly agonists, are typically used as host directed therapeutics according to the present invention. As such, prostanoid receptor modulators of the invention, preferably prostanoid receptor agonists, typically have host-directed activity against an NTM.

Prostanoids, including prostacyclin, are vasoactive eicosanoids which act upon both innate immune cells (including macrophages, neutrophils, and Natural Killer cells), and adaptive immune cells (including T-and B-cells). Therefore, the host-directed therapeutic effect of a prostanoid receptor modulator of the invention, particularly a prostanoid receptor agonist of the invention, may be mediated by such innate and/or adaptive immune cells. Typically, the host-directed therapeutic effect of a prostanoid receptor modulator of the invention is mediated by cells of the innate immune system, particularly monocytes and/or macrophages. As described herein, typically prostanoid receptor modulators of the invention act by binding to prostanoid receptors, particularly EP₂, EP₁, EP₃, FP, TP, IP and/or DP₁, preferably EP₂. Thus, the host-directed therapeutic effect of a prostanoid receptor modulator of the invention, particularly a prostanoid receptor agonist, may be mediated by binding of said prostanoid receptor modulator to a prostanoid receptor (e.g. EP₂, EP₁, EP₃, FP, TP, IP and/or DP₁, preferably EP₂) on cells of the innate and/or adaptive immune system, particularly innate immune cells, preferably monocytes and/or macrophages.

Without being bound by theory, during NTM infection increased expression levels of some prostanoid receptors, such as EP₂, may decrease susceptibility to NTM infection. Thus, increased signalling through prostanoid receptors, such as EP₂, is believed to have a protective effect. Accordingly, prostanoid receptor agonists may be used to increase signalling through such prostanoid receptors (particularly EP₂ signalling by EP₂ agonists) during NTM infection and may be particularly useful at treating and/or preventing NTM infection. Preferably said prostanoid receptor agonists bind to EP₂ receptor.

NTMs are often intrinsically drug-resistant due to their thick, impermeable cell walls and/or their presence in granulomas, which effectively decrease drug uptake. NTMs can also express proteins that specifically target clinically used antibacterial compounds, including drug efflux pumps and drug deactivating enzymes such as B-lactamases. NTMs can also acquire drug resistance mechanisms.

A prostanoid receptor modulator of the invention, preferably a prostanoid receptor agonist, may be used to treat drug-resistant NTM, whether drug resistance is intrinsic to said NTM and/or acquired. A prostanoid receptor modulator of the invention, preferably a prostanoid receptor agonist, may be used to treat multi-drug resistant NTM. Without being bound by theory, as exemplified herein, the effect of prostanoid receptor modulators, particularly prostanoid receptor agonists, against NTMs is host-directed (i.e. host-mediated). Therefore, this enables prostanoid receptor modulators to effectively treat NTM infections, despite any drug-resistance of the NTM(s) in question.

### Treatment Outcomes

“Treatment” according to the present invention may be defined as providing a treatment outcome as defined below. These definitions may apply to therapeutic and prophylactic treatments as described herein.

As described herein, the invention relates to the use of prostanoid receptor modulators for the treatment and/or prevention of NTM infection. Preferably, the prostanoid receptor modulator is a prostanoid receptor agonist as described herein. Preferably, the prostanoid receptor agonist is an EP₂ receptor agonist. More preferably, the prostanoid receptor agonist is a prostacyclin analogue which binds to the EP₂ receptor.

As demonstrated herein, treatment with a prostanoid receptor modulator of the invention, preferably a prostanoid receptor agonist, may result in killing of the NTMs. As such, treatment with a prostanoid receptor modulator of the invention may reduce a patient's bacterial load. Accordingly, treatment may reduce a patient's NTM load by at least about 10%, at least about 20%, at least about 25%, at least about 30%, at least about 40%, at least about 50% or more. Preferably, there is a reduction in a patient's NTM load by at least 30%, more preferably at least 40%.

Alternatively or additionally, treatment with a prostanoid receptor modulator of the invention, preferably a prostanoid receptor agonist, may reduce NTM survival rate, typically by at least about 10%, at least about 20%, at least about 25%, at least about 30%, at least about 40%, at least about 50% or more. Preferably, there is a reduction in the NTM's survival rate of at least 30%, more preferably at least 40%.

Alternatively or additionally, treatment with a prostanoid receptor modulator of the invention, preferably a prostanoid receptor agonist, may improve a patient's quality of life (QOL). This may involve improving/reducing symptoms of NTM infection. Symptom reduction may be complete or partial, given that NTM infection can result in underlying structural damage to the lungs). QOL may be assessed using any standard QOL assessment, of which examples are known in the art. Non-limiting examples include the St. George Respiratory Questionnaire (SGRQ), the EuroQOL Five Dimensions Questionnaire, and the Medical Outcomes Short-Form-36 Questionnaire (SF-36).

Alternatively or additionally, treatment with a prostanoid receptor modulator of the invention, preferably a prostanoid receptor agonist, may decrease the number and/or size of NTM nodules within the lungs. The effectiveness of treatment with a prostanoid receptor modulator of the invention may be assessed by radiographic monitoring (including x-ray, Computed tomographic (CT) and ¹⁸F-fluorodeoxyglucose-PET imaging). By way of non-limiting example, treatment with a prostanoid receptor modulator of the invention may improve a patient's radiograph scoring of the number, size of NTM nodules within the lungs and/or volume of diseased tissue.

Alternatively or additionally, treatment with a prostanoid receptor modulator of the invention, preferably a prostanoid receptor agonist, may decrease the concentration of biomarkers associated with NTM infection. These biomarkers may be quantified in any appropriate sample from a patient, such as blood or sputum. Such biomarkers include both NTM and/or or host biomarkers. Non-limiting examples of such biomarkers include serum anti-glycopeptidolipid IgA (anti-GPL IgA) and carbohydrate antigen 19-9 (CA 19-9).

Alternatively or additionally, treatment with a prostanoid receptor modulator of the invention, preferably a prostanoid receptor agonist, may increase the concentration of biomarkers associated with the patient's immune response to NTM infection. These biomarkers may be quantified in any appropriate sample from a patient, such as blood or sputum. Such biomarkers may include both NTM and/or or host biomarkers, typically host biomarkers. Non-limiting examples of such biomarkers include IL-17 and IL-23.

Alternatively or additionally, treatment with a prostanoid receptor modulator of the invention, preferably a prostanoid receptor agonist, may decrease the time taken for a patient to be clear of detectable NTM infection. This may be quantified as time to negative culture, whereby samples are taken from a patient at different time points and cultured for NTM growth. Treatment according to the invention may decrease the time taken for a sample to be NTM negative on culture. Treatment according to the present invention may decrease the time to negative culture by at least 1 week, at least 2 weeks, at least 3 weeks, at least 4 weeks, at least 6 weeks, at least 8 weeks, at least 12 weeks, or more.

Alternatively or additionally, treatment with a prostanoid receptor modulator of the invention, preferably a prostanoid receptor agonist, may decrease the time to culture conversion. Culture conversion may be based on assessment of monthly sputum cultures, with culture conversion achieved if a patient had three consecutive monthly negative sputum cultures, with all sputum samples collected at each visit required to be culture-negative. Treatment according to the present invention may decrease the time to culture conversion by at least 4 weeks, at least 6 weeks, at least 8 weeks, at least 12 weeks or more.

Alternatively or additionally, treatment with a prostanoid receptor modulator of the invention, preferably a prostanoid receptor agonist, may increase the distance achieved in the 6-minute-walk test (6MWT) by at least 10 m, at least 15 m, at least 20 m, at least 25 m or more.

Alternatively or additionally, treatment with a prostanoid receptor modulator of the invention, preferably a prostanoid receptor agonist, may improve other measures of patient functional activity.

Alternatively or additionally, treatment with a prostanoid receptor modulator of the invention, preferably a prostanoid receptor agonist, may decrease the duration of intravenous antibiotic treatment required. Treatment according to the present invention may decrease the duration of intravenous antibiotic treatment required by at least 1 week, at least 2 weeks, at least 3weeks, at least 4 weeks, at least 6 weeks, at least 8 weeks, or more.

Alternatively or additionally, treatment with a prostanoid receptor modulator of the invention, preferably a prostanoid receptor agonist, may decrease the total duration of antibiotic treatment required. Treatment according to the present invention may decrease the duration of antibiotic treatment required by at least 1 week, at least 2 weeks, at least 3 weeks, at least 4 weeks, at least 6 weeks, at least 8 weeks, or more.

Alternatively or additionally, treatment with a prostanoid receptor modulator of the invention, preferably a prostanoid receptor agonist, may decrease the number of antibiotics required for a combination regime to treat NTM infection. By way of non-limiting example, if without a prostanoid receptor modulator of the invention a combination regime requires three antibiotics, then a prostanoid receptor modulator of the invention may decrease the number of antibiotics required for a combination regime to one or two of these antibiotics.

Alternatively or additionally, treatment with a prostanoid receptor modulator of the invention, preferably a prostanoid receptor agonist, may improve a patient's respiratory function. The improvement in lung function may be quantified by any appropriate means. Standard lung function parameters are known in the art and include vital capacity (VC), forced vital capacity (FVC) and forced expiratory volume (FEV) (e.g. FEV in 1 second, FEV₁).

As described herein, one of the most serious clinical consequences of NTM infection is that the presence of such an infection can be a contraindication in patients requiring a lung transplant, such as those with underlying chronic respiratory disorders. Therefore, a particular advantage of the use of prostanoid receptor modulator of the invention, preferably a prostanoid receptor agonist, is that such treatment may reduce or remove a patient's contraindication for other therapies, such as a lung transplant. In other words treatment with a prostanoid receptor modulator of the invention may maintain a patient's eligibility for additional treatments. Such additional treatments include transplants, particularly lung transplants. Treatment with a prostanoid receptor modulator of the invention may maintain a patient's transplant eligibility, preferably lung transplant eligibility.

Treatment according to the present invention may result in any combination of the treatment outcomes as described herein. Typically treatment according to the invention results in a treatment outcome or combination of treatment outcomes that is associated with a reduction in clinical risk.

The effectiveness of a treatment of the invention may be compared with a suitable control, examples of which are described herein. By way of non-limiting example, one or more treatment outcome in an individual treated according to the present invention may be compared with a suitable control, such as the same parameter in healthy individual, or the parameter in an individual (typically the same individual) with NTM infection prior to treatment.

As described herein, a prostanoid receptor modulator (preferably an agonist), composition or drug delivery system of the invention may be used in combination with one or more additional active ingredient or therapeutic for the treatment and/or prevention of NTM infection. Thus a prostanoid receptor modulator, preferably a prostanoid receptor agonist, may be used in combination with a second therapy for NTM infection. A prostanoid receptor modulator may be combined with any one or more additional active agent or second therapy, provided said one or more additional active agent or second therapy is appropriate for use in the treatment of NTM infection. The terms “second therapy” and “one or more additional active agent” are used interchangeably herein.

The use of a combination of a prostanoid receptor modulator, preferably a prostanoid receptor agonist, with one or more additional activate agent may provide advantages compared with treatment with either the prostanoid receptor modulator and/or the one or more additional active agent when administered as sole therapies. Thus, the advantages may be achieved compared with (i) the prostanoid receptor modulator administered as a sole therapy; and/or (ii) the second therapy administered as a sole therapy. Non-limiting examples of these advantages are set out below. Any one or more of these advantages may be achieved in any combination using a combination treatment of the invention.

When a prostanoid receptor modulator of the invention, preferably a prostanoid receptor agonist, is used in combination with the second therapy for NTM infection, the combination treatment may reduce the number of agents required for the second therapy. By way of non-limiting example, if the second therapy is a standard antibiotic therapy requiring three antibiotics, then a prostanoid receptor modulator of the invention may be used in combination with one or two of these antibiotics.

When a prostanoid receptor modulator of the invention, preferably a prostanoid receptor agonist, is used in combination with the second therapy for NTM infection, the dose (or concentration) of the second agent may be reduced. By way of non-limiting example, if the second therapy requires administration of azithromycin at a concentration of 10 mg/kg/dose, then a dose of 5 mg/kg/dose azithromycin may be used in combination with a prostanoid receptor modulator of the invention.

When a prostanoid receptor modulator of the invention, preferably a prostanoid receptor agonist, is used in combination with the second therapy for NTM infection, the combination treatment may reduce the duration of treatment required for the second therapy, particularly the duration of intravenous administration of the second therapy. By way of non-limiting example, if the second therapy is a standard antibiotic therapy of 12 weeks' duration (e.g. by i.v. administration), then a prostanoid receptor modulator of the invention may be used in combination with the standard therapy administered (e.g. by i.v. administration) for 6 weeks.

When a prostanoid receptor modulator of the invention, preferably a prostanoid receptor agonist, is used in combination with the second therapy for NTM infection, the combination treatment may reduce or avoid the need to use additional agents which require intravenous (i.v.) administration in the second therapy. By way of non-limiting example, if the second therapy is a standard antibiotic requiring i.v. administration of two antibiotics and oral administration of a third, then combining with a prostanoid receptor modulator of the invention may allow one of the i.v. antibiotics to instead by administered orally, or for an alternative antibiotic to be used which can be administered by a non-i.v. route. Preferably when a prostanoid receptor modulator of the invention is used in combination with the second therapy for NTM infection, each agent within the combination treatment may be administered orally.

When a prostanoid receptor modulator of the invention, preferably a prostanoid receptor agonist, is used in combination with the second therapy for NTM infection, the combination treatment may reduce the number, duration and/or severity of one or more side effects associated with the second therapy. This may be independent on or dependent on other advantages (e.g. following from a reduced concentration and/or number of additional agents being used). By way of non-limiting example, if the second therapy causes nausea, skin rashes and jaundice in a patient, then combining a prostanoid receptor modulator of the invention with the standard therapy may reduce or eliminate jaundice and/or nausea in the patient.

Combining a prostanoid receptor modulator of the invention, preferably a prostanoid receptor agonist, with a second therapy for NTM infection, may reduce the likelihood of a patient being admitted to hospital and/or may reduce the duration of their stay in hospital if admittance is required. This reduction in the likelihood and/or duration of hospital admittance may be greater than the reduction in the likelihood and/or duration of hospital admittance achieved when (i) the prostanoid receptor modulator is used as a sole therapy; and/or (ii) the second therapy is administered as a sole therapy.

When a prostanoid receptor modulator of the invention, preferably a prostanoid receptor agonist, is used in combination with the second therapy for NTM infection, the combination treatment may improve the patient's QOL. Suitable Questionnaires for QOL assessment are known in the art and non-limiting examples are described herein. This improvement may be greater than the improvement in QOL achieved when (i) the prostanoid receptor modulator is used as a sole therapy; and/or (ii) the second therapy is administered as a sole therapy.

When a prostanoid receptor modulator of the invention, preferably a prostanoid receptor agonist, is used in combination with the second therapy for NTM infection, the combination treatment may maintain a patient's respiratory function (i.e. prevent further deterioration in a patient's respiratory function), or even improve a patient's respiratory function. By “maintaining a patient's respiratory function”, it is meant that there is no significant decrease in the patient's respiratory function compared with treatment with the second therapy alone. By way of non-limiting example, a patient's respiratory function may be no more than 1.0-fold lower, no more than 0.5-fold lower, no more than 0.25-fold lower, or less than a patient's respiratory function when treated with the second therapy alone. The term “maintaining a patient's respiratory function” may be defined such that the patient's respiratory function is statistically unchanged (e.g. p<0.05, p<0.01) compared with the patient's respiratory function when treated with the second therapy alone. Preferably, the combination treatment may improve a patient's respiratory function. This improvement may be greater than the improvement in lung function achieved when (i) the prostanoid receptor modulator is used as a sole therapy; and/or (ii) the second therapy is administered as a sole therapy. The improvement in lung function may be quantified by any appropriate means. Standard lung function parameters are known in the art and include vital capacity (VC), forced vital capacity (FVC) and forced expiratory volume (FEV) (e.g. FEV in 1 second, FEV₁).

When a prostanoid receptor modulator of the invention, preferably a prostanoid receptor agonist, is used in combination with the second therapy for NTM infection, the combination treatment may reduce or remove a patient's contraindication for other therapies, such as a transplant, particularly a lung transplant. Thus, when a prostanoid receptor modulator of the invention is used in combination with the second therapy for NTM infection, the combination treatment may maintain a patient's eligibility for additional treatments. Such additional treatments include transplants, particularly lung transplants.

### Therapy

The invention provides a prostanoid receptor modulator (preferably an agonist), compositions comprising said prostanoid receptor modulator, drug delivery systems for delivering said prostanoid receptor modulator for use in the treatment and/or prevention of NTM infection. Said prostanoid receptor modulator may modulate one or more prostanoid receptor directly or indirectly. Typically prostanoid receptor modulator of the invention directly modulate one or more prostanoid receptor. Preferably said direct prostanoid receptor modulators bind to one or more prostanoid receptor as described herein. Preferably said prostanoid receptor modulators are prostanoid receptor agonists such as prostacyclin analogues. More preferably, said prostanoid receptor agonists bind to the EP₂ receptor.

The term “treat” or “treating” as used herein encompasses prophylactic treatment (e.g. to prevent onset of NTM infection) as well as corrective treatment (treatment of an individual already suffering from NFM infection). Preferably, the term “treat” or “treating” as used herein means corrective treatment. The term “treat” or “treating” encompasses treating both NTM infection, symptoms thereof and diseases/disorder associated therewith. In some embodiments the term “treat” or “treating” refers to a symptom of an NTM infection.

The “treatment” may be defined as providing a treatment outcome as defined herein, or any combination thereof.

A prostanoid receptor modulator (preferably an agonist), composition or drug delivery system of the invention may be used in the treatment of an individual having a NTM infection as described herein. An individual may be screened for an NTM infection prior to treatment (e.g. using a radiographic imaging, or detecting and/or quantifying NTM within a sample, such as a sputum sample), and may be selected for treatment based on presence of an NTM infection.

A “therapeutically effective amount” is any amount of a prostanoid receptor modulator (preferably an agonist), composition or drug delivery system of the invention which, when administered alone or in combination to a patient for treating NTM infection or a symptom thereof or a disease associated therewith is sufficient to provide such treatment of the NTM infection, or symptom thereof, or associated disease.

A “prophylactically effective amount” is any amount of a prostanoid receptor modulator (preferably an agonist), composition or drug delivery system of the invention that, when administered alone or in combination to an individual inhibits or delays the onset or reoccurrence of NTM infection, or a symptom thereof or disease associated therewith). The prophylactically effective amount may prevent the onset or reoccurrence of NTM infection entirely. “Inhibiting” the onset means either lessening the likelihood of NTM infection onset (or symptom thereof or disease associated therewith) or preventing the onset entirely.

The terms “subject”, “individual” and “patient” are used interchangeably herein to refer to a mammalian individual. Generally, the individual may be human; in other words, in one embodiment, the “individual” is a human. The individual may not have been previously diagnosed as having NTM infection (or symptom thereof or disease associated therewith). Alternatively, the individual may have been previously diagnosed as having NTM infection (or symptom thereof or disease associated therewith). The individual may also be one who exhibits disease risk factors, or one who is asymptomatic for NTM infection (or symptom thereof or disease associated therewith). The individual may also be one who is suffering from or is at risk of developing NTM infection (or symptom thereof or disease associated therewith).

Administration of a prostanoid receptor modulator, composition or drug delivery system of the invention may be by any appropriate route. Non-limiting examples of conventional routes include inhalation; intraperitoneal, intravenous, intra-arterial, subcutaneous, and/or intramuscular injection; infusion; rectal, vaginal, topical and oral administration. The most appropriate administration route may be selected based on the prostanoid receptor modulator, composition or drug delivery system to be used. Typically the prostanoid receptor modulator, composition or drug delivery system of the invention may be administered by inhalation, particularly oropharyngeal inhalation, oral inhalation and/or nasal inhalation, or by oral, intravenous or intra-arterial administration. Preferably the prostanoid receptor modulator, composition or drug delivery system of the invention is for inhalation, intravenous, subcutaneous injection or oral administration, more preferably inhalation, even more preferably oral inhalation. Preferably said prostanoid receptor modulators are prostanoid receptor agonists.

By way of non-limiting example, a prostanoid receptor modulator, such as Treprostinil when administered by i.v. infusion may be administered at a rate of from about 0.5 ng/kg/min to about 200 ng/kg/min, such as from about 0.625 ng/kg/min to about 50 ng/kg/min, from about 1.25 ng/kg/min to about 100 ng/kg/min from about 50 ng/kg/min to about 160 ng/kg/min.

It will be appreciated by one of skill in the art that the appropriate dosage of a prostanoid receptor modulator, composition or drug delivery system of the invention, can vary from individual to individual. Determining the optimal dosage will generally involve the balancing of the level of therapeutic benefit against any risk or deleterious side effects. The selected dosage level will depend on a variety of factors including, the route of administration, the severity of the individual's/patient's fibrosis, and the species, sex, age, weight, condition, general health, and prior medical history of the individual/patient. By way of non-limiting example, orally administered Treprostinil may be administered at a dose of 0.125 mg three times daily (TID), or 0.25 mg twice daily (BID). These doses can be titrated by 0.125 mg TID or 0.25/0.5 mg BID every 3 to 4 days to the highest tolerated dose. By way of further non-limiting example, Treprostinil administered by inhalation may be administered at a dose of 18 μg four times daily (TID). This dose can be titrated by 6 μg or 12 μg four TID if a dose of 18 μg 4 TID is not tolerated. Preferably said prostanoid receptor modulators are prostanoid receptor agonists.

The frequency of dosing selected may also be dependent on a range of factors. The skilled person will be able to select the most suitable dosing regimen appropriate for the individual. Typically, a prostanoid receptor modulator, composition or drug delivery system of the invention is administered between about once every three months to about four times per day. For example, the prostanoid receptor modulator, composition or drug delivery system of the invention may be administered once every three months, once per month, twice per month, once per week, twice per week, 3 times per week, 4 times per week, 5 times per week, 6 times per week, once a day, twice a day, 3 times per day, 4 times per day or more. Preferably, the prostanoid receptor modulator, composition or drug delivery system of the invention is administered twice a day (BID) or three times a day (TID). Treatment may be continued for at least one month, at least two months, at least four months, at least six months, at least one year, at least two years, at least five years, at least ten years or more, including indefinite treatment/treatment for the life of an individual. Preferably said prostanoid receptor modulators are prostanoid receptor agonists.

A prostanoid receptor modulator, composition or drug delivery system of the invention may have a treatment outcome as defined herein within 8-52 weeks (preferably within 36 weeks, more preferably within 24 weeks, even more preferably within 12 weeks) from baseline. Preferably, administration of the prostanoid receptor modulator, composition or drug delivery system of the invention may provide a treatment outcome within 36 weeks, more preferably within 24 weeks, even more preferably within 12 weeks. Preferably said prostanoid receptor modulators are prostanoid receptor agonists.

The treatment outcome may be sustained (e.g. maintained) subsequent to and/or during treatment for several weeks or months or years. A prostanoid receptor modulator, composition or drug delivery system of the invention may provide a sustained treatment outcome for at least 5, 10, 12, 16, 18, 20, 22, 24, 38, 32, 36, 40, 52, 78 or 104 weeks. For example, administration of a prostanoid receptor modulator, composition or drug delivery system of the invention may provide a sustained treatment outcome for at least 5 weeks, at least 10 weeks, at least 20 weeks, or at least 52 weeks.

A prostanoid receptor modulator, composition or drug delivery system of the invention may be used in combination with one or more additional active ingredient or therapeutic for the treatment and/or prevention of NTM infection. In other words, the prostanoid receptor modulator, composition or drug delivery system of the invention may be used in combination with a second therapy for NTM infection. By way of non-limiting example, the second therapy for NTM infection may be an antibiotic and/or protein kinase inhibitor. Thus, the prostanoid receptor modulator, composition or drug delivery system of the invention may be used in combination with an antibiotic and/or a protein kinase inhibitor. Examples of antibiotics used in the treatment of NTM infection are known in the art, and it is within the routine practice of one of ordinary skill in the art to select a suitable antibiotic for combining with a prostanoid receptor modulator of the invention. Non-limiting examples of antibiotics that may be used in combination with a prostanoid receptor modulator of the invention include a carbapenem antibiotic, one or more of amikaicin, azithromycin, clarithromycin, erythromycin, imipenem, tigecycline, cefoxitin, moxifloxacin, minocycline, co-trimoxazole, linezolid, isoniazid, rifampicin, ethambutol, and pyrazinamide. Non-limiting examples of protein kinase inhibitors that may be used in combination with a prostanoid receptor modulator of the invention include gefitinib, erlotinib and imatinib. Preferably said prostanoid receptor modulators are prostanoid receptor agonists.

The one or more additional active ingredient or therapeutic may be administered sequentially (before or after) the prostanoid receptor modulator, composition or drug delivery system of the invention. The one or more additional active ingredient or therapeutic may be administered simultaneously with the prostanoid receptor modulator, composition or drug delivery system of the invention. Preferably said prostanoid receptor modulators are prostanoid receptor agonists.

The invention also provides a prostanoid receptor modulator, composition or drug delivery system of the invention for use in a method of treating a NTM infection (or symptom thereof or disease associated therewith). Preferably said prostanoid receptor modulators are prostanoid receptor agonists.

The invention also provides a method for the treatment or prevention of a NTM infection (or symptom thereof or disease associated therewith), comprising administering a prostanoid receptor modulator, composition or drug delivery system of the invention. Said method typically comprises administering a therapeutically effective amount of a prostanoid receptor modulator to a patient in need thereof. Preferably said prostanoid receptor modulators are prostanoid receptor agonists.

The invention also provides a prostanoid receptor modulator, composition or drug delivery system of the invention for use in the manufacture of a medicament for the treatment or prevention of a NTM infection (or symptom thereof or disease associated therewith). Preferably said prostanoid receptor modulators are prostanoid receptor agonists.

As demonstrated herein, treatment of a NTM infection with a prostanoid receptor modulator according to the invention may be more efficacious that treatment of a corresponding NTM infection with antibiotic therapy. Treatment of a NTM infection with a prostanoid receptor modulator according to the invention may be more efficacious that treatment of a NTM infection with a carbapenem antibiotic, such as imipenem. Treatment of a NTM infection with a prostanoid receptor modulator according to the invention may at least about 10%, at least about 20%, at least about 25%, at least about 30%, at least about 40%, at least about 50% or more efficacious than treatment of a corresponding NTM infection with antibiotic therapy, preferably carbapenem therapy, more preferably imipenem therapy. Preferably, said prostanoid receptor modulator is a prostanoid receptor agonist.

Preferably, adjunctive therapy with a prostanoid receptor modulator of the invention and one or more antibiotic may be more efficacious than therapy with (i) the antibiotic therapy alone; or (ii) the prostanoid receptor modulator alone. Adjunctive therapy with a prostanoid receptor modulator of the invention and one or more carbapenem antibiotic may be more efficacious that treatment of a NTM infection said one or more carbapenem antibiotic. Said one or more carbapenem antibiotic consist or comprise imipenem. Adjunctive therapy with a prostanoid receptor modulator of the invention and one or more antibiotic (e.g. one or more carbapenem antibiotic) may be at least about 10%, at least about 20%, at least about 25%, at least about 30%, at least about 40%, at least about 50% or more efficacious than treatment of a corresponding NTM infection with antibiotic therapy alone, preferably carbapenem therapy alone, more preferably imipenem therapy alone. Preferably said prostanoid receptor modulator is a prostanoid receptor agonist.

A prostanoid receptor modulator, composition or drug delivery system of the invention may be used to treat NTM infection in a patient who has one or more pre-existing disease or disorder, or a patient with an NTM infection who has no underlying conditions or pre-existing diseases or disorders. Preferably, a prostanoid receptor modulator, composition or drug delivery system of the invention may be used to treat NTM infection in a patient who has one or more pre-existing disease or disorder. The term “pre-existing disorder” is used herein to refer to a disease or disorder which was present (whether symptomatic or asymptomatic) before the NTM infection. Therefore, any disease or disorder may a pre-existing disease or disorder if it is present before the patient acquires an NTM infection. Preferably said prostanoid receptor modulator is a prostanoid receptor agonist.

Given the nature of NTM infections, a pre-existing disease or disorder may typically be a disease or disorder of the lungs or respiratory tract, particularly chronic diseases or disorders of the lungs or respiratory tract. Non-limiting examples of chronic respiratory diseases include chronic obstructive pulmonary disease (COPD), asthma, occupational lung diseases such as pneumoconiosis, pulmonary hypertension (PH), bronchiectasis and cystic fibrosis (CF).

Alternatively or in addition, the pre-existing disease or disorder may be an immunodeficiency. The immunodeficiency may be a primary immunodeficiency or a secondary immunodeficiency (also referred to as an acquired immunodeficiency). Non-limiting examples of secondary immunodeficiencies include those caused by (i) infections such as HIV; (ii) cancer, e.g. leukaemia or lymphoma; and/or (iii) immunosuppressive agents, such as chemotherapeutics, corticosteroids (e.g. glucocorticoids), immunosuppressants such as methotrexate and azathioprine; and/or (iv) environmental toxins such as heavy metals.

Patients with an indwelling prosthetic and/or indwelling prosthetic material are also at increased risk of NTM infection and pathology resulting from such infections. Therefore, alternatively or in addition, a patient to be treated according to the invention may have an indwelling prosthetic and/or indwelling prosthetic material. Non-limiting examples of indwelling prosthetic and/or indwelling prosthetic material include joint prostheses, bone grafts, vascular grafts, dental prostheses, ossicular prostheses, intravascular catheters, mechanical heart valves, urinary catheters, orthopaedic implants and indwelling medical devices.

Patients who have undergone open heart surgery are also susceptible to NTM infections. In particular, some heater-coller devices used during open heart surgery have been found to be associated with/risk factors for NTM infection. Therefore, alternatively or in addition, a patient to be treated according to the invention may have undergone open heart surgery. NTM are slow-growing bacteria and infections may take months to develop. NTM infections cases associated with heater-coller devices and/or open heart surgery may be diagnosed months and up to several years after an open heart surgery involving such heater-cooler devices. Therefore, the time of initiation and/or duration of treatment according to the invention is not particularly limited.

In some preferred embodiments, a patient to be treated for a NTM infection according to the present invention has a chronic respiratory disease, optionally CF, COPD, or PH. Any NTM infection may be treated in such patients, particularly an M. abscessus infection.

In particular, the invention relates to the treatment of CF patients with NTM infections, particularly M. abscessus infections.

A patient to be treated according to the invention may also be infected with M. tuberculosis. In other words, a patient to be treated according to the invention may have tuberculosis (TB).

Preferably, a patient to be treated according to the invention may not be infected with M. tuberculosis. In other words, a patient to be treated according to the invention may not have tuberculosis (TB).

### Compositions and Formulations

The invention provides compositions, particularly pharmaceutical compositions, comprising a prostanoid receptor modulator, composition or drug delivery system of the invention and a pharmaceutically acceptable excipient, diluent, adjuvant, immunoregulatory agent and/or antimicrobial compound. Preferably said prostanoid receptor modulator is a prostanoid receptor agonist such as a prostacyclin analogue. More preferably, said prostanoid receptor agonist is a prostacyclin analogue which binds to the EP₂ receptor.

The prostanoid receptor modulator (preferably a prostanoid receptor agonist) may be in the form of a pro-drug, analogue, derivative, or salt as described herein.

Compositions or formulations comprising a prostanoid receptor modulator (preferably an agonist), composition or drug delivery system of the invention may further comprise one or more additional active ingredient or therapeutic, such as an antibiotic or protein kinase inhibitor as described herein. The prostanoid receptor modulator, composition or drug delivery system of the invention and the one or more additional active ingredient or therapeutic may be provided as a kit of parts. Preferably said prostanoid receptor modulator is a prostanoid receptor agonist.

As described herein, administration of immunogenic compositions, therapeutic formulations, medicaments and prophylactic formulations is generally by conventional routes, with inhalation and particularly oropharyngeal inhalation, being preferred.

Formulation of a prostanoid receptor modulator (preferably an agonist), composition or drug delivery system of the invention may therefore be adapted using routine practice to suit the preferred route of administration.

Formulations suitable for distribution as aerosols are preferred, and it would be routine for one of ordinary skill in the art to prepare such formulations.

By way of further non-limiting example, a prostanoid receptor modulator (preferably an agonist), composition or drug delivery system of the invention, compositions or therapeutic/prophylactic formulations and/or medicaments thereof may be prepared as injectables, either as liquid solutions or suspensions. Solid forms suitable for solution in, or suspension in, liquid prior to injection may alternatively be prepared. The preparation may also be emulsified, or the encapsulated in liposomes or microcapsules. Liposomal and/or nanoparticle formulation and/or administration may be preferred. By way of non-limiting example, the prostanoid receptor modulator may be formulated in liposomes comprising or consisting of dipalmitoylphosphatidylcholine and cholesterol, in any appropriate w/w ratio (e.g. 2:1 w/w). The prostanoid receptor modulator, composition or drug delivery system of the invention may also be formulated as a dry-powder formulation.

The active immunogenic ingredients are often mixed with excipients which are pharmaceutically acceptable and compatible with the active ingredient. Suitable excipients are, for example, water, saline, dextrose, glycerol, ethanol, or the like and combinations thereof. In addition, if desired, the composition may contain minor amounts of auxiliary substances such as wetting or emulsifying agents, pH buffering agents, and/or adjuvants which enhance the effectiveness of the prostanoid receptor modulator.

Generally, the carrier is a pharmaceutically-acceptable carrier. Non-limiting examples of pharmaceutically acceptable carriers include water, saline, and phosphate-buffered saline. In some embodiments, however, the composition is in lyophilized form, in which case it may include a stabilizer, such as BSA. In some embodiments, it may be desirable to formulate the composition with a preservative, such as thiomersal or sodium azide, to facilitate long term storage.

Examples of buffering agents include, but are not limited to, sodium succinate (pH 6.5), and phosphate buffered saline (PBS; pH 6.5 and 7.5).

Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders.

### Screening Assays

The present inventors are the first to demonstrate that prostanoid receptor modulators, particularly prostanoid receptor agonists, can be used to treat NTM infection. As exemplified herein, the inventors have also shown that expression of certain prostanoids and prostanoid receptors are increased in individuals who are less susceptible to mycobacterial infection. In particular the inventors have also shown that expression of prostacyclin and EP₂ is increased in individuals who are less susceptible to mycobacterial infection.

Therefore, quantifying these changes in an in vitro setting has the potential to identify patients who have altered (increased or decreased) levels of different prostanoids and/or prostanoid receptors, and hence are suitable for treatment according to the present invention.

Accordingly, the invention provides a method of screening a patient for suitability for a treatment according to the invention. Said method may comprise (i) determining the level of one or more prostanoid receptor in a patient; and/or (ii) determining the level of a prostanoid or prostanoid metabolite in a patient. Typically said method comprises determining the level of one or more prostanoid receptor in a patient.

Determining the level of a prostanoid or prostanoid metabolite in a patient may comprise determining the level of prostacyclin or a prostacyclin metabolite, e.g. 6-keto-PGF1-α. Any appropriate technique may be used to determine the level of the prostanoid or prostanoid metabolite. Suitable techniques are known in the art and within the routine practice of one of ordinary skill in the art. Non-limiting examples include mass spectrometry.

Determining the expression level of one or more prostanoid receptor may be done at the gene level (e.g. by detecting and/or quantifying the level of prostanoid receptor mRNA) or the protein level. Any appropriate technique may be used to determine the expression level of the one or more prostanoid receptor. Suitable techniques are known in the art and within the routine practice of one of ordinary skill in the art. Non-limiting examples for quantifying gene expression include RNASeq, northern blotting, ribonuclease protection assay, RT-PCR and real-time RT-PCR. Non-limiting examples for quantifying protein expression include Western blotting and ELISA.

A patient may be identified as suitable for treatment according to the invention when (i) the expression level of one or more prostanoid receptor crosses a threshold level; and/or (ii) the amount of prostanoid or prostanoid metabolite crosses a threshold level.

The threshold level may be dependent on the particular one or more prostanoid receptor and/or the one or more prostanoid or prostanoid metabolite. As described herein, some prostanoids and prostanoid receptors (e.g. EP₂) are increased in individuals with low susceptibility to NTMs. For treatment comprising administering a modulator of one or more such prostanoid receptors, a patient may be identified as suitable for treatment according to the invention if their expression level of said one or more prostanoid receptor is above a threshold level By way of non-limiting example, a patient may be identified as suitable for treatment with a EP₂ modulator such as Treprostinil if they are determined to have EP₂ expression above a threshold level.

In particular the inventors have also shown that expression of prostacyclin and EP₂ is increased in individuals who are less susceptible to mycobacterial infection.

Accordingly, the invention provides a method of screening a patient for suitability for a treatment of the invention, comprising: (a) determining the expression level of a prostanoid receptor in a sample from the patient; and/or (b) determining the amount of a prostanoid or prostanoid metabolite (e.g. 6-keto-PGF1-α) in a sample from the patient; and identifying the patient as suitable for said treatment when the expression level of a prostanoid receptor and/or the amount of a prostanoid or prostanoid metabolite (e.g. 6-keto-PGF1-α) crosses a threshold level.

The prostanoid receptor may be one or more of EP₂, EP₁ and/or EP₃ receptor, preferably EP₂. In some preferred embodiments, gene expression of the prostanoid receptor is determined, optionally the expression of one or more of PTGER2, PTGER1 and/or PTGER3, preferably PTGER2.

The expression (gene and/or protein) of the one or more prostanoid receptor and/or the amount of the prostanoid or prostanoid metabolite (e.g. 6-keto-PGF1-α) may be determined in a sample obtained from a patient. Any appropriate sample may be used, e.g. sputum, blood (including whole blood, plasma and/or serum), bronchoalveolar lavage (BAL), urine, stool, or other tissue or fluid samples, such as CSF and biopsy samples.

The invention also provides a method for identifying an agent which may be suitable for the treatment of NTM infection. Said method may comprise the steps of: (a) culturing cells in vitro; (b) contacting said cells with an NTM; (c) adding a test agent to the cultured cells; and (d) determining a change in phenotype, for example an increase in killing of NTM, in response to the test agent.

Any type of test agent may be employed in a method of the invention. The skilled person will be familiar with the various types of test agents which may be added to cultured cells in vitro. The test agent may be any type of prostanoid receptor modulator as described herein. By way of non-limiting example, the test agent may be a small molecule, a nucleic acid agent (for example, an siRNA, a plasmid, an antisense oligonucleotide or a nucleic acid aptamer), an antibody or antibody-fragment thereof, or a peptide aptamer. Compositions or drug delivery systems comprising or expressing a test agent may also be employed in a screening method of the invention. Any disclosure herein in relation to drug delivery systems and compositions applies equally and without limitation for drug delivery systems and compositions comprising or expressing test agents for use in a screening method of the invention. By way of non-limiting example, a liposomal drug delivery system comprising a test agent may be used.

Any cell type capable of being cultured in vitro may be utilised in a screening method of the invention. Typically, the cells are primary cells (i.e. cells derived from animal tissues) or cell lines. The cells used in a method of the invention may be a cell type involved in NTM infection and the immune response to such infections. By way of non-limiting example, the cells may be monocytes or macrophages, typically human monocytes or macrophages such as the THP-1 cell line.

The cells may be derived from an individual to be treated, i.e. from an individual with an existing NTM infection. The cells may be derived from a biopsy sample of an individual with an NTM infection. Cells may be isolated from a biopsy sample using flow cytometry.

The change in phenotype may be compared with a control. Any appropriate control may be used, and it is within the standard competency of one of ordinary skill in the art to select an appropriate control. Examples of suitable controls are described herein. For example, a control may be a population of the same cell type (preferably from the same source), wherein the control cells are cultured in the same conditions as the cells exposed to the test agent, composition or drug delivery system, but wherein the control cells are not exposed to the test agent, composition or drug delivery system.

A screening method of the invention may consist of the steps described herein (carried out in sequentially in the described order), or may comprise additional steps. Non-limiting example of additional steps include isolating and/or the cells after exposure to the test agent, composition or drug delivery system.

### EXAMPLES

The invention is now described with reference to the Examples below. These are not limiting on the scope of the invention, and a person skilled in the art would be appreciate that suitable equivalents could be used within the scope of the present invention. Thus, the Examples may be considered component parts of the invention, and the individual aspects described therein may be considered as disclosed independently, or in any combination.

### Example 1—Expression of 6-Keto-PGF1-α is Increased in Children With Low Susceptibility to Mycobacterial Infection

To investigate whether early eicosanoid inflammatory responses to mycobacteria in school age children correlates with early elimination of mycobacteria and a protective host response, a case-control study was conducted comparing pairs of Gambian children older than 5 years of age with the same sleeping proximity to the same adult with smear-positive pulmonary tuberculosis but discordant tuberculin skin test result status. We recruited 29 pairs of children from such households and conducted a whole blood in vitro infection assay using a recombinant strain of BCG.

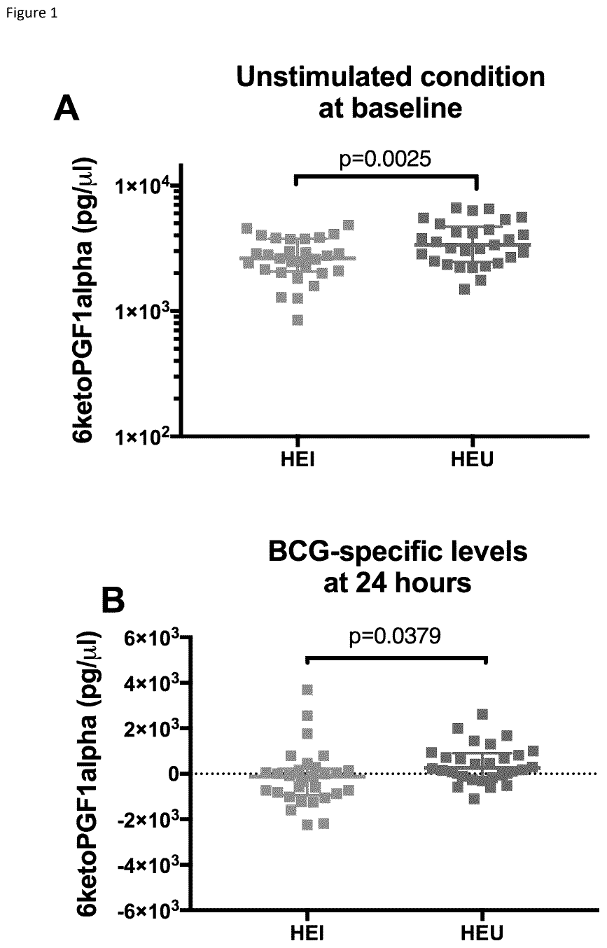

Supernatants from baseline, 24 hours and 96 hours were analysed using an ultrahigh-performance liquid chromatography-electrospray ionization triple quadrupole mass spectrometry targeted eicosanoid assay. As shown in FIG. 1, samples from children who had high TB exposure but were uninfected (HEU) showed significantly higher levels of 6-keto-PGF1-α, a prostacyclin metabolite, than children with matched high TB exposure but infection (HEI) in baseline unstimulated samples (HEU 3,352, Interquartile range (IQR) 2,451→4,694; ((HEI median 2,627 pg/μl, IQR 2,056 to 3,751; p=0.0025) and BCG-specific levels at 24 hours (HEU 258.6 pg/μl, IQR −162.5 to 906; HEI median −127.2 pg/μl, IQR −948.2 to 224.7; p=0.00379).

In contrast, testing the samples for levels of PGE2 did not reveal a significant difference between HEI and HEU children: baseline unstimulated samples: HEU median 111 pg/μl, IQR 81 to 152; HEI median 109.1 pg/μl, IQR 80 to 160; p=0.90; BCG-specific levels at 24 hours: HEU median −360 pg/μl, IQR −1503 to 727.5; HEI median −1178, IQR −2772 to −374, p=0.099) (data not shown).

### Example 2—Expression of PTGER2 is Increased in Children With low Susceptibility to Mycobacterial Infection

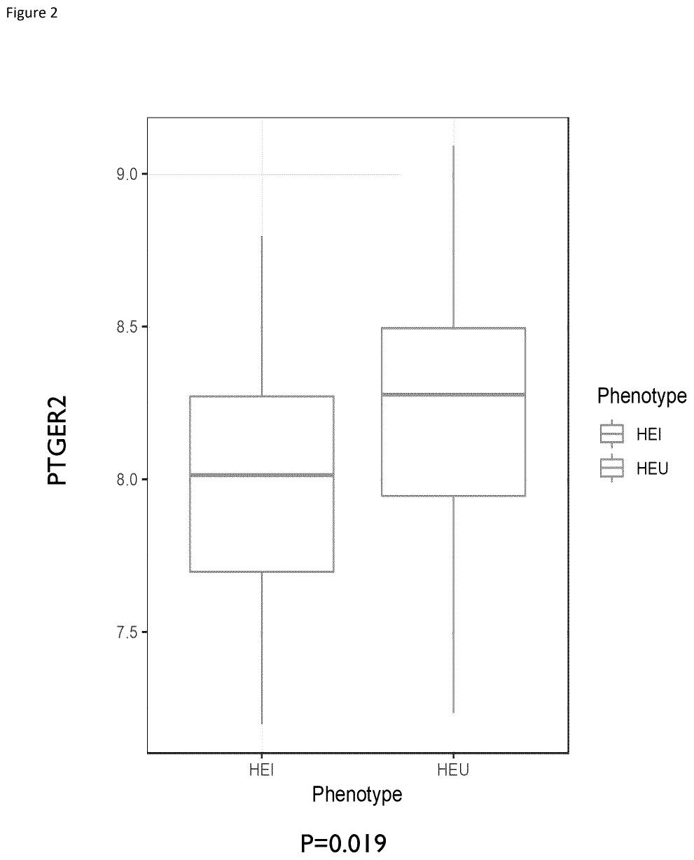

RNASeq analysis was also conducted on unstimulated whole blood samples from the same group of children as in Example 1, and a hypothesis-testing matched analysis applied to examine expression levels of genes in the prostacyclin pathway. As shown in FIG. 2, expression of the transcript PTGER2, encoding an isoform (Ensembl Accession No. ENSG00000125384, version 103.38, accessed 9 Jul. 2022) of prostanoid receptor (EP2) that binds both PGE2 and prostacyclin analogues was also significantly higher in HEU compared to HEI children (p=0.019, mean of differences 0.23).

Example 3—Expression of PTGER2 is Increased in Adults With Latent TB Infection in Response to Mycobacterial Challenge

Available gene expression data for Vietnamese adults with TB infection was interrogated to establish whether genes in the prostacyclin pathway were also differentially expressed in these patients in response to in vitro mycobacterial infection.

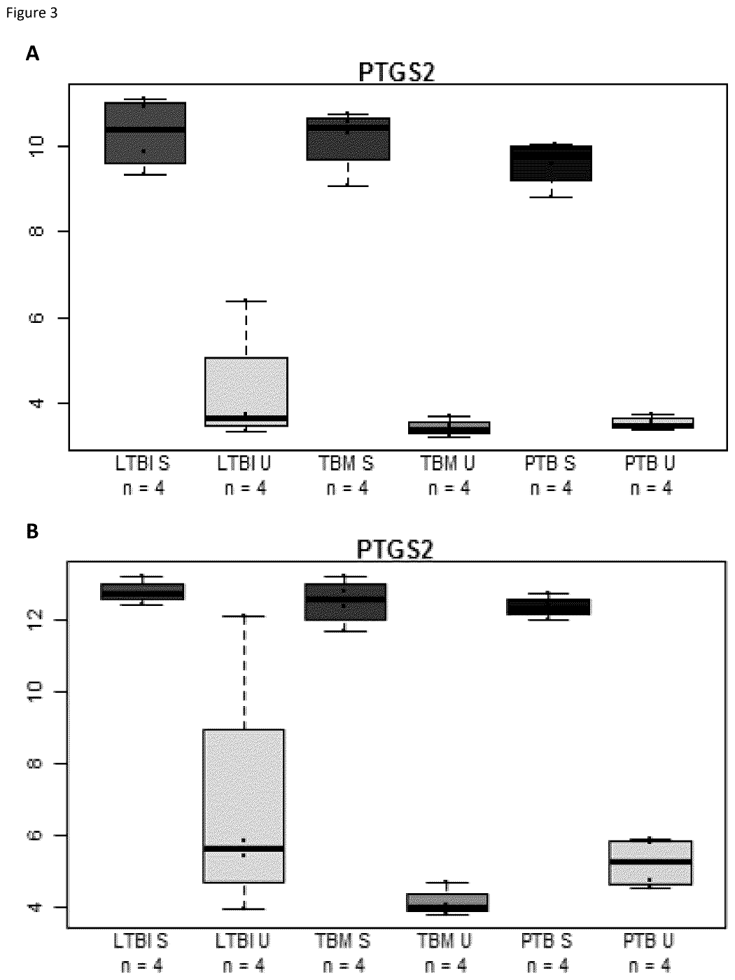

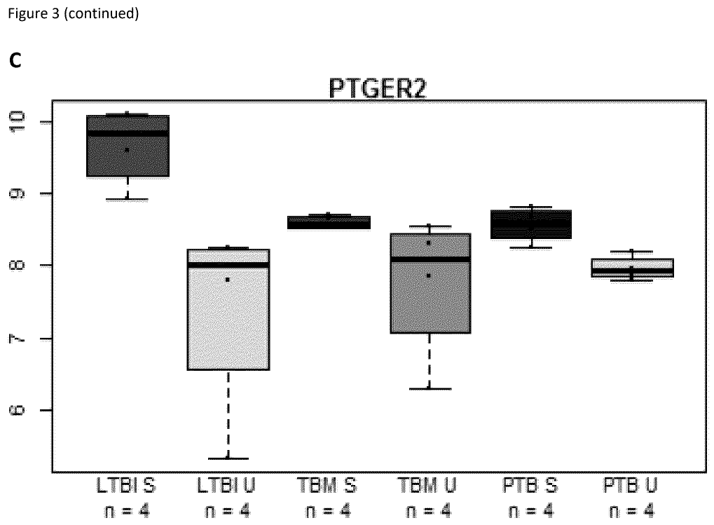

M. tuberculosis infection of monocyte-derived macrophages was found to increase expression of PTGS2, and PTGER2 (logFoldChange 0.356, p=0.03, FIG. 3) in patients with TB infection, particularly latent TB infection (LTBI). PTGS2 is the inducible isoform of cyclooxygenase upstream of prostacyclin synthesis.

### Example 4—The Prostacyclin Pathway is Implicated in Host-NTM Interactions

To determine whether the prostacyclin pathway is implicated in host-mycobacterial interactions with NTMs, NTM infection of zebrafish was investigated. Prostacyclin synthase (PTGIS) was found to be upregulated. Further, there was differential regulation of the two zebrafish EP2 receptors in the granuloma compared to healthy macrophages (PTGIS, 4.3-fold increased expression, p=0.02; PTGER2a, 5-fold decreased expression, p=0.001; PTGER2b, 5-fold increased expression, p=0.0003; data not shown).

### Example 5—Treprostinil Stimulates Killing of M. abscessus by THP-1 Monocytes

To confirm functional relevance of the findings in Example 4, Treprostinil, a licensed prostacyclin analogue that acts on the EP2 receptor and that is used in the treatment of pulmonary arterial hypertension, was added during in vitro infection of the monocytic human cell line, THP1, with two human mycobacterial pathogens.

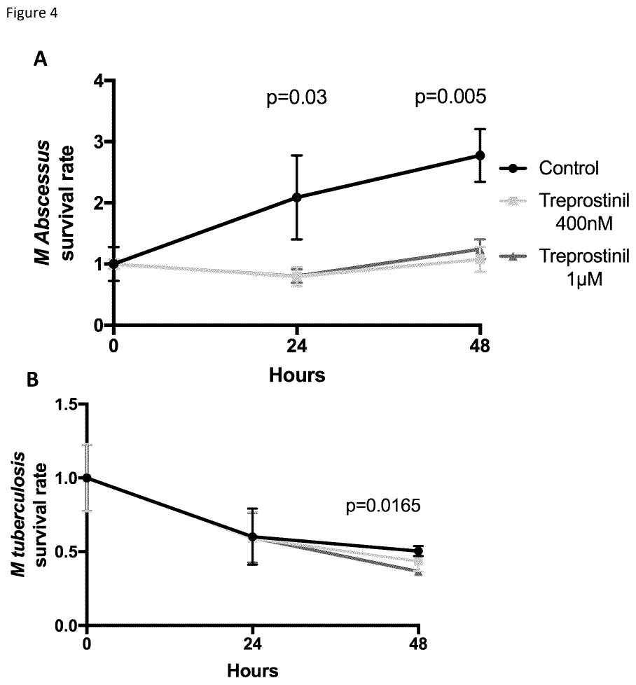

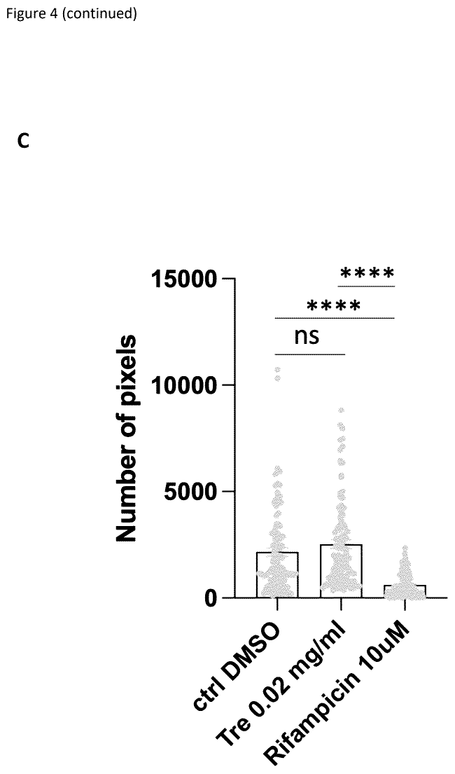

Mycobacterial killing was enhanced for both M. abscessus—(survival rates at 48H—control: 2.7, 400 nM: 1.07, 1 μM: 1.24, p=0.005, FIG. 4A) and M. tuberculosis—control: 0.50, 400 nM: 0.44, 1 μM: 0.37, p=0.0165, FIG. 4B). However, the effect on M. abscessus was observably greater than that on M. tuberculosis. This suggests that Treprostinil has significantly greater therapeutic potential in the treatment of M. abscessus and other NTM compared with M. tuberculosis.

The effectiveness of treating NTM infection with Treprostinil compared with the treatment of M. tuberculosis-like mycobacteria was further investigated using M. marinum in zebrafish, as M. marinum is a model organism for TB in zebrafish. As shown in FIG. 4C, treatment with Treprostinil had no observable effect on M. marinum in zebrafish, whereas conventional therapy with rifampicin significantly reduced M. marinum numbers. Together with the minimal effect on M. tuberculosis shown in FIG. 4B, this further supports the conclusion that Treprostinil is effective in killing NTM, but not M. tuberculosis or M. tuberculosis-like mycobacteria such as M. marinum in zebrafish.

### Example 6—Treprostinil Killing of M. Abscessus is Dose-Dependent, and Treprostinil is More Effective in Killing of M. Abscessus Than Gold-Standard Carbapenem Antibiotic Imipenem

To further investigate the therapeutic potential of Treprostinil, we compared the activity of Treprostinil against M abscessus monotherapy with imipenem in a zebrafish model. Imipenem is a carbapenem antibiotic that is recommended for use in drug resistant tuberculosis (DR-TB) when an effective regimen with combination of other second line drugs is not possible.

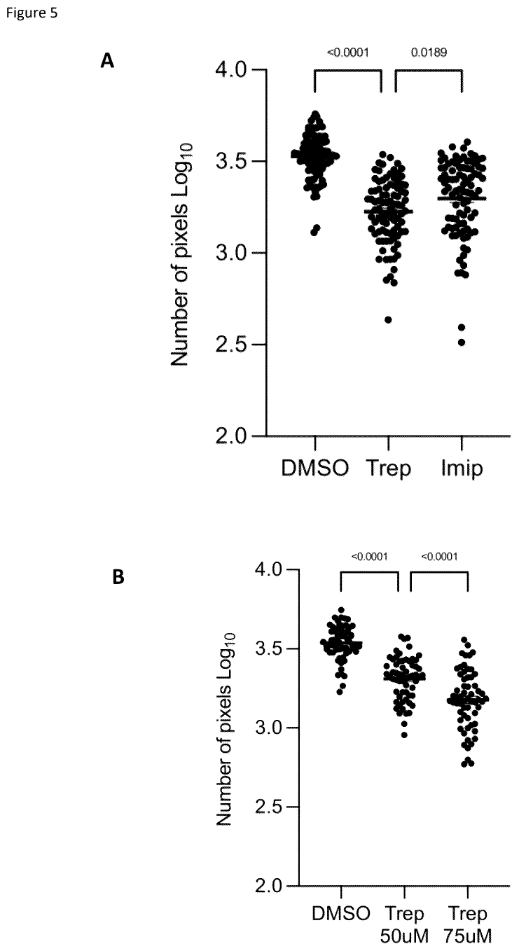

As shown in FIG. 5A, Treprostinil was significantly more effective than imipenem in driving killing of M abscessus. Thus, whilst Treprostinil had no effect against M. marinum in zebrafish, where it a M. tuberculosis-like mycobacterium and a model organism for TB, Treprostinil was effective against the NTM M. abscessus.

Furthermore, a dose response to the effect was also observed (FIG. 5B), supporting the conclusion that Treprostinil is a potent agent in treating M abscessus infection.

### Example 7—Treprostinil Killing of M. Abscessus is a not an Antibiotic Effect

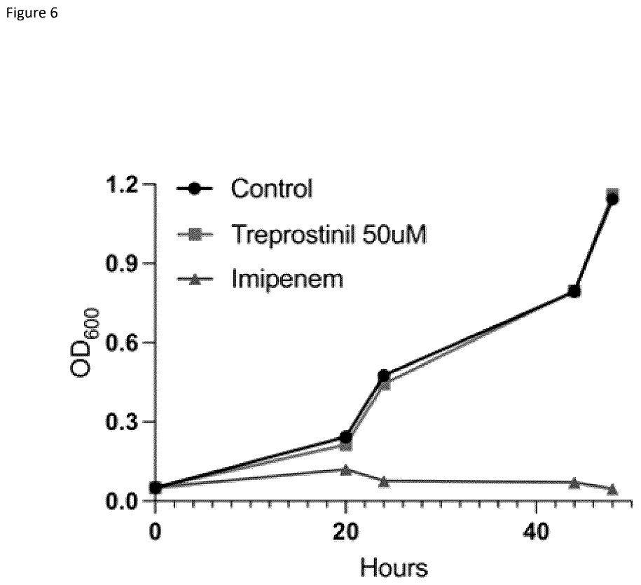

To test whether the effect of Treprostinil against M. abscessus observed in Example 5 was an off-target direct antibiotic effect, Treprostinil was added to M. abscessus in liquid culture (i.e. in the absence of THP1 cells). As shown in FIG. 6, M. abscessus treated with Treprostinil in this way demonstrated no difference in growth compared with the untreated M. abscessus, which demonstrates that the effect of Tresprostinil is not an antibiotic effect, and suggests a potential host-mediated effect (as proven in Example 8 below). In contrast and as expected, treatment of M. abscessus with imipenem has a bactericidal effect against M abscessus in the absence of THP1 cells (data not shown).

### Example 8—The Effect of Treprostinil Against M. Abscessus is Mediated by the EP2 Receptor

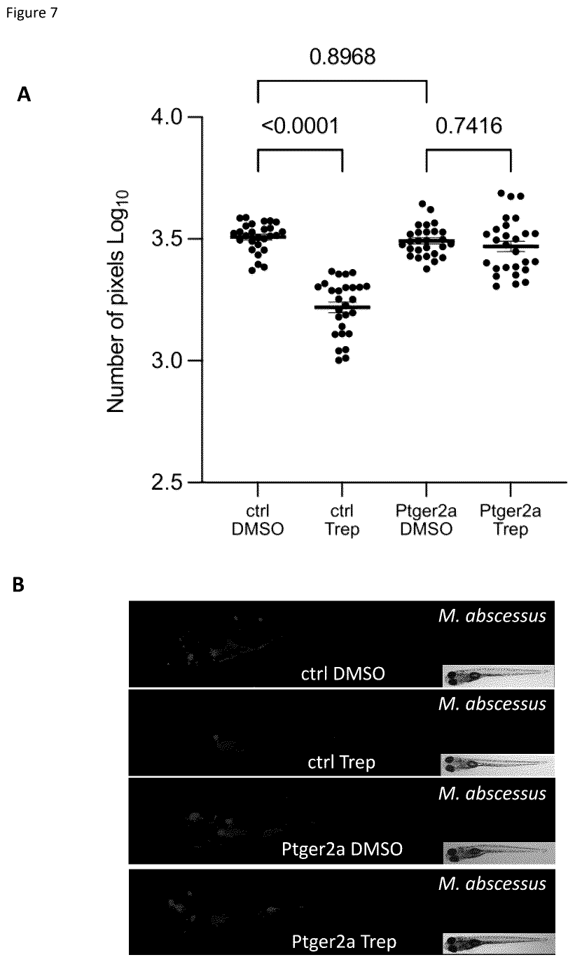

To explore the mechanism of action of Treprostinil in this context, the zebrafish homolog of the EP2 receptor was knocked out using CRISPR and control and KO zebrafish were infected with M. abscessus. As shown in FIG. 7, EP2a KO completely abrogated Treprostinil rescue. This definitively confirms that Treprostinil acts via an EP2-mediated host-directed therapeutic effect.

### Discussion

The experiments herein investigate natural paediatric immunity to mycobacterial infections by studying expression data for children exposed but uninfected with M tuberculosis. Matching for exposure to the same adult TB index case controls for risk factors for infection.

Prostacyclin metabolites were found higher in Gambian children who are exposed but uninfected at baseline and in response to BCG at 24 hours. Further, RNASeq from these same children shows differential expression of the EP₂ receptor.

Treprostinil, a clinically licenced prostacyclin analogue has been demonstrated to enhance killing of M. abscessus. This effect is host mediated via action upon the EP2a receptor, and was more effective as a sole therapy than gold-standard imipenem treatment. In contrast, Treprostinil was observed to have a minimal effect on M. tuberculosis, and no effect on M. marinum, a M. tuberculosis-like mycobacteria infecting zebrafish. Therefore, these results demonstrate that Treprostinil is effective in killing NTM, but not M. tuberculosis or M. tuberculosis-like mycobacteria.

In cystic fibrosis (CF) and bronchiectasis patients, NTM infection, including M. abscessus, is increasing in prevalence and associated with rapidly worsening disease. It is also difficult to treat, requiring antibiotic regimens with substantial side effects, including severe nausea, deafness, and impaired liver function. Lung transplantation is often contraindicated in the presence of NTM and mortality is high. Therefore, there is an urgent unmet clinical need to provide safe and effective treatments for NTM infection.

The experiments reported herein demonstrate that prostanoid receptor modulators, including existing licensed drugs such as Treprostinil have potential clinical utility as host-directed adjunctive therapy for NTM infection. This represents an entirely novel approach to adjuvant therapy to treat antimicrobial resistant NTM infections with potential to improve effectiveness, shorten treatment duration and reduce the number and/or toxicities of antimicrobial agents currently required.

1. A method for treating or preventing a nontuberculous mycobacterial infection comprising administering a therapeutically effective amount of a prostanoid receptor agonist to a patient in need thereof.

2. The method of claim 1, wherein the prostanoid receptor agonist comprises:
(a) an EP₂ receptor agonist; or
(b) an EP₂ receptor agonist which binds to the EP₂ receptor.

3. The prostanoid receptor agonist for use of claim 1, wherein the prostanoid receptor agonist comprises:
(a) a prostacyclin analogue; or
(b) a non-prostanoid prostanoid receptor agonist.

4. The method of claim 3, wherein the prostacyclin analogue is selected from the group consisting of:
(a) Treprostinil, Iloprost, Epoprostenol, Beraprost and Cisaprost and
(b) Treprostinil.

5. The method of claim 3, wherein the non-prostanoid prostanoid receptor agonist is selected from the group consisting of Selexipag, MRE-269, Ralinepag (APD811), Esuberaprost and ONO-1301.

6. The method of claim 1, wherein the nontuberculous mycobacterial infection is a nontuberculous mycobacteria (NTM) selected from the group consisting of Mycobacterium abscessus, Mycobacterium avium Complex (MAC), Mycobacterium chelonae, Mycobacterium fortuitum, Mycobacterium kansasii, Mycobacterium scrofulaceum, Mycobacterium smegmatis, Mycobacterium ulcerans, Mycobacterium xenopi and Mycobacterium bovis Bacillus Calmette-Guerin (BCG).

7. The method of claim 1, wherein the prostanoid receptor agonist has host-directed activity against the NTM.

8. The method of claim 7, wherein the host-directed effect is mediated by monocytes, macrophages or macrophages and monocytes.

9. The method of claim 1, wherein the NTM comprises a drug-resistant NTM.

10. (canceled)

11. The method of claim 1, wherein prostanoid receptor agonist is administered by inhalation, intravenous administration, subcutaneous injection or oral administration.

12. The method of claim 1, wherein the method further comprises administration of a second therapy for nontuberculous mycobacterial infection.

13. The method of claim 12, wherein the second therapy for nontuberculous mycobacterial infection is an antibiotic.

14. The method of claim 13, wherein the antibiotic is selected from the group consisting of:
(a) a carbapenem antibiotic;
(b) one or more antibiotics selected from the group consisting of amikacin, azithromycin, clarithromycin, erythromycin, imipenem, isoniazid, rifampicin, ethambutol, and pyrazinamide, and
(c) one or more of amikacin, azithromycin, clarithromycin, erythromycin, imipenem or rifampicin.

15. The method of claim 13, wherein the administration of the prostanoid receptor agonist and the second therapy for nontuberculous mycobacterial infection:
(a) reduces the duration of hospital admission of the individual in need thereof;
(b) reduces the dose of administered second therapy agents required for the treatment;
(c) reduces the duration of intravenous administration of the administered second therapy;
(d) reduces the need for requirement for intravenous administration of the administered second therapy;
(e) reduces one or more side effect associated with administration of the second therapy;
(f) improves patient quality of life as measured using the St. George Respiratory Questionnaire (SGRQ);
(g) improves patient respiratory function;
(h) maintains eligibility for additional treatments;
(i) maintains eligibility for a transplant; or
(j) maintains eligibility for a lung transplant.

16. The method of claim 15, wherein the individual in need thereof a has a pre-existing disease or disorder.

17. The method of claim 16, wherein the individual in need thereof has:
(a) a chronic lung disease;
(b) a primary immunodeficiency, or an acquired or secondary immunodeficiency;
(c) an indwelling prosthetic or indwelling prosthetic material; or
(d) undergone open heart surgery.

18. The method of claim 16, wherein:
(a) the patient has cystic fibrosis or bronchiectasis, or an M. abscessus infection;
(b) treatment with the prostanoid receptor agonist maintains the patient's eligibility for a lung transplant that would otherwise have been contraindicated; or
(c) a patient to be treated does not have tuberculosis.

19 and 20 (canceled)

21. A method of treating a nontuberculous mycobacterial infection, comprising administering a therapeutically effective amount of a therapeutic combination of drugs consisting of: a prostanoid receptor agonist and an antibiotic.

22. (canceled)

23. The method of claim 1, wherein the individual in need thereof is selected by a method comprising:
(a) determining the amount of 6-keto-PGF1-α in a sample from a patient; and/or
(b) determining the expression level of a prostanoid receptor in a sample from the patient; and
(c) identifying the patient as suitable for said treatment when the amount of 6-keto-PGF1-α or the expression level of a prostanoid receptor is above a threshold level,
and optionally the prostanoid receptor is EP₂; or gene expression of PTGER2 is determined.

24. (canceled)

25. The method of claim 13, wherein administration of the prostanoid receptor agonist and antibiotic provides an adjunctive therapy which is more efficacious than administration of (i) the antibiotic therapy alone, or (ii) the prostanoid receptor agonist alone.

26. The method of claim 9, wherein the drug-resistant NTM comprises a multi-drug resistant NTM.
